(19) United States
(12) Patent Application Publication
(10) Pub. No.: US 2026/0294226 A1
(43) Pub. Date: Oct. 1, 2026

(54) CHARACTERIZING CHANNEL CLEANING EFFICACY

(71) Applicant: Saban Ventures Pty Limited, Macquarie Park, NSW (AU)

(72) Inventors: Boris SHARFF, Hornsby, NSW (AU); Amir Hossein KAYVANPOUR, Carlingford, NSW (AU); Neil Douglas Alan ROGERS, Lane Cove, NSW (AU)

(21) Appl. No.: 19/480,618

(22) PCT Filed: May 24, 2024
(86) PCT No.: PCT/IB2024/055092

§ 371 Date: Oct. 31, 2025

(30) Foreign Application Priority Data
May 26, 2023 (AU) 2023901646

(51) Int. Cl. A61B 1/12; G01N 29/028

(52) U.S. Cl. CPC A61B 1/125; G01N 29/028

(57) ABSTRACT

Presented herein are techniques for cleaning the interior/internal lumens (e.g., channels, cylinders, valve sockets and connectors, etc.) of an apparatus, such as a medical device (medical instrument), using contaminant-detaching fluidic compositions. In particular, the techniques presented herein introduce an apportioned amount of a contaminant-detaching fluidic composition into an interior lumen of a medical device. The contaminant-detaching fluidic composition is configured, and propelled through at least a portion of the interior lumen device, so that the contaminant-detaching fluidic composition can clean the walls of the interior lumen (e.g., interact with the walls of interior lumen to remove contaminants there from).

### CROSS-REFERENCE TO RELATED APPLICATIONS

This application claims priority to and the benefit of Australian Provisional Patent Application No. 2023901646, entitled “Sensor system and method for characterizing a cleaning process,” filed May 26, 2023, which is hereby incorporated for reference in its entirety for all purposes.

### BACKGROUND

### Technical Field

The present disclosure generally relates to techniques for characterizing a cleaning efficacy of a channel cleaning process during cleaning of, for example, a channel of a medical device.

### Related Art

There are several different types of systems/devices that include interior conduits/channels/tubes (generally and collectively referred to herein as channels). The channels can include, for example, dental lines, food/drink lines, medical channels of different medical devices (medical instruments), etc. Medical devices, in particular, can include channels that can be used to perform diagnostic and/or surgical procedures. For example, an endoscope is a medical device that includes channels that can be used to visually inspect hollow organs or body cavities, deliver/extract fluids, etc. Specially designed endoscopes are used for different examinations, such as bronchoscopy, cystoscopy, gastroscopy, and proctoscopy. Additionally, many such systems/devices/components are designed to be reused. Therefore, proper cleaning is desirable before said reuse.

### SUMMARY

In one aspect, a method is provided herein. The method comprises: delivering a cleaning fluid flow through a lumen of a medical device during a channel cleaning process; obtaining a vibration measurement while the cleaning fluid flow passes through the lumen; and characterizing a cleaning efficacy of the channel cleaning process based on the vibration measurement.

In another aspect, a system configured to perform a channel cleaning process is provided. The system comprises: a mechanism to deliver a cleaning fluid through at least one lumen of an apparatus; at least one transducer operably coupled to the at least one lumen and configured to measure interaction between the cleaning fluid and the at least one lumen as the cleaning fluid passes through the at least one lumen; and at least one processor configured to use the interaction measured by the at least one transducer to characterize a cleaning efficacy of the channel cleaning process.

In another aspect, a method is provided herein. The method comprises: delivering a cleaning fluid flow through a lumen of a medical device; obtaining a vibration measurement while the cleaning fluid flow passes through the lumen; characterizing a level of cleaning efficacy after the cleaning fluid flow passes through the lumen; and associating the vibration measurement with the level of cleaning efficacy.

### BRIEF DESCRIPTION OF THE DRAWING

Embodiments of the present disclosure are described herein in conjunction with the accompanying drawings, in which:

FIG. 1 is a schematic diagram illustrating an endoscope having channels with which the cleaning techniques presented herein can be implemented.

FIG. 2A is a schematic diagram illustrating a process for cleaning a channel and for characterizing cleaning efficacy of a cleaning process, in accordance with certain embodiments of the present disclosure.

FIG. 2B is a schematic diagram of a cleaning efficacy analysis system, in accordance with certain embodiments of the present disclosure.

FIG. 3 is a schematic diagram illustrating a process for characterizing cleaning efficacy for multiple channels, in accordance with certain embodiments of the present disclosure.

FIGS. 4, 5, 6, and 7 illustrate various systems for cleaning an interior surface of a channel of a medical device using a cleaning fluid, in accordance with certain embodiments of the present disclosure.

FIG. 8 illustrates example graphs corresponding to measurements obtained by various sensors during a channel cleaning process.

FIGS. 9A and 9B illustrate example vibration measurements obtained by a vibration sensor over a period of time.

FIGS. 10A and 10B illustrate example quantities of vibration measurements having certain frequencies.

FIG. 11 illustrates a flowchart of a method for cleaning a channel of a medical device and characterizing cleaning efficacy, in accordance with certain embodiments of the present disclosure.

FIG. 12 illustrates a flowchart of a method for cleaning a channel of a medical device based on a cleaning efficacy, in accordance with certain embodiments of the present disclosure.

FIG. 13 illustrates a flowchart of a method for associating a vibration measurement with a level of cleaning efficacy, in accordance with certain embodiments of the present disclosure.

FIG. 14 illustrates a block diagram of an example control sub-system for use in characterizing cleaning efficacy, in accordance with certain embodiments of the present disclosure.

### DETAILED DESCRIPTION

As noted, there are a number of different types of systems/devices that include interior channels (conduits/tubes), such as dental lines, food/drink lines, medical lumens, etc., that may need periodic and/or regular cleaning. There are also a number of different techniques that perform this channel cleaning by passing some form of cleaning fluid through the channel. For example, co-owned International Patent Publication No. WO2022256871A1, the content of which is hereby incorporated by reference herein, describes a channel cleaning system in which a cleaning fluid in the form of a liquid-powder is passed through the device channels (e.g., lumens).

Presented herein are techniques for monitoring cleaning of interior surfaces of a channel during a cleaning process, such as that described in co-owned International Patent Publication No. WO2022256871A1. More specifically, in accordance with embodiments presented herein, a cleaning fluid is delivered to (through) a channel (e.g., through the lumen defined by the channel). While the cleaning fluid passes through the channel, the cleaning efficacy of the channel cleaning process is characterized. As used herein, reference to “characterizing” a cleaning efficacy of a channel cleaning process can refer to determining, evaluating, monitoring, defining, and/or analyzing the efficacy of the channel cleaning process in whole or part, including at a current time/stage, an entirety of the channel cleaning process, portions of the channel cleaning process, such as the efficacy of a given cleaning fluid flow used during a channel cleaning process, etc.

In certain embodiments, a transducer (e.g., vibration sensor) is operably coupled to the channel and is configured to obtain a measurement of interaction (interaction measurement) between the cleaning fluid and the channel as the cleaning fluid passes there through. The interaction measurement can be, in turn, used to characterize a cleaning efficacy of the cleaning process (e.g., the efficacy of a given cleaning fluid flow). In certain examples, the interaction measurement is a vibration measurement.

For ease of description, the techniques presented herein are primarily described with reference to cleaning the interior surfaces of specific medical channels, namely the interior surfaces of endoscope channels. However, it is to be appreciated that the techniques presented herein can also or alternatively be used to clean the interior surface of any type of channel. Before describing further details of the systems and methods presented herein, a brief description of endoscopes and endoscope channels is provided below.

An endoscope is an elongate tubular medical device that may be rigid or flexible and which incorporates an optical or video system and light source. Typically, an endoscope is configured so that one end can be inserted into the body of a patient via a surgical incision or via one of the natural openings of the body. Internal structures near the inserted end of the endoscope can thus be viewed by an external observer.

As well as being used for investigation, endoscopes are also used to conduct diagnostic and surgical procedures. Endoscopic procedures are increasingly popular as they are minimally invasive in nature and provide a better patient outcome (through reduced healing time and exposure to infection), enabling hospitals and clinics to achieve higher patient turnover.

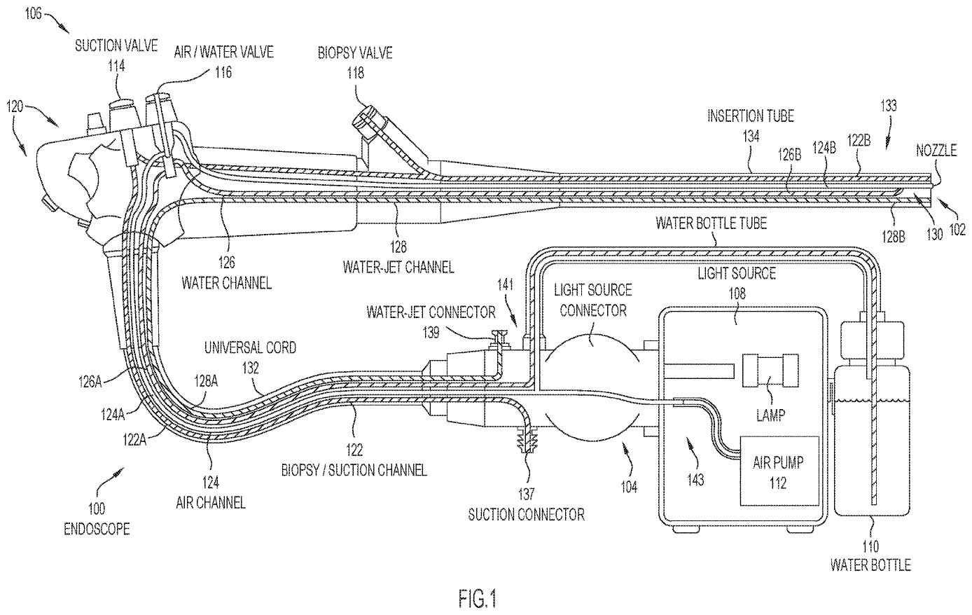

FIG. 1 is a schematic diagram of an example endoscope 100 with which aspects of the techniques presented herein can be implemented. As shown, endoscope 100, similar to most endoscopes, has a long tube-like structure with a distal end (distal tip) 102 at one end for insertion into a patient and an opposing proximal or connector end 104, with a control handle 106 located between the two ends (e.g., generally at the center of the length between connector end 104 and distal end/tip 102). The connector end 104 includes a plurality of connectors that enable the endoscope to be attached to, for example, a light source 108, water source 110, a suction source (not shown in FIG. 1), and a pressurized air source 112. For example, shown in FIG. 1A, is a suction port/connector 137, a water-jet (auxiliary) port/connector 139, a water port/connector 141, and an air-port/connector 143. The control handle 106 is held by the operator during the procedure to control the endoscope 100 via valves, which include in this example a suction valve 114, an air/water cylinder (valve) 116, and a biopsy valve 118, and control wheels 120.

As shown in FIG. 1, endoscope 100 includes internal channels used either for delivering air and/or water, providing suction or allowing access for forceps and other medical equipment required during the procedure. As such, the distal tip 102 contains the camera lens (not shown in FIG. 1), and the exits for the lighting, air, and water, as well as exits for suction and forceps. Some of the internal channels run from one end of the endoscope 100 to the other, while others run via valve sockets at the control handle. Some channels bifurcate while and others join from two into one.

More specifically, shown in FIG. 1 is a biopsy/suction channel 122, an air channel 124, a water channel 126, and a water-jet channel 128. The biopsy/suction channel 122 includes two sections, referred to as proximal section 122A and distal section 122B that are connected via the suction valve 114. The air channel 124 also includes two sections, referred to as proximal section 124A and distal section 124B that are connected via the air/water cylinder (valve) 116. Similarly, the water channel 126 also includes two sections, referred to as proximal section 126A and distal section 126B that are connected via the air/water cylinder 116. The distal section 126B of the water channel joins to the distal section 124B of the air channel at a location 130 within the distal section 133 of the endoscope and each exit via nozzle 149. The water-jet channel 128 extends directly from the connector end 104 to the distal end 102 (via the control handle 106) but is similarly referred to as having a proximal section 128A and distal section 128B. The proximal sections 122A, 124A, 126A, and 128A of the channels are sometimes referred to as being located within a universal cord section (cord) 132 of the endoscope 100, while the distal sections 122B, 124B, 126B, and 128B of the channels are sometimes referred to as being located within an insertion tube 134 of the endoscope. More generally, as used herein, the proximal sections 122A, 124A, 126A, and 128A are the portions of the channels located between the connector end 104 and a valve (e.g., valve 114 or 116) at the control handle 106 and/or a mid-point of the control handle 106, as applicable. The distal sections 122B, 124B, 126B, and 128B are the portions of the channels located between the valve (e.g., valve 114 or 116) at the control handle 106 and/or a mid-point of the control handle 106, and the distal end 102 of the endoscope 102.

The high cost of endoscopes means they must be re-used. As a result, because of the need to avoid cross infection from one patient to the next, each endoscope must be thoroughly cleaned and disinfected or sterilized after each use. This involves the cleaning of not only the outer of the endoscope 100, but also cleaning and disinfecting the internal channels (e.g., channels 122, 124, 126, and 128 of FIG. 1).

Endoscopes used for colonoscopic procedures are typically between 2.5 and 4 meters long and have one or more channels of diameter of no more than a few millimeters. Ensuring that such long narrow channels are properly cleaned and disinfected between patients presents a considerable challenge. The challenge of cleaning is also made more difficult by the fact that there is not just one configuration/type of endoscope. Indeed, there are a variety of endoscopic devices, each suited to a particular insertion application, such as colonoscopes inserted into the colon, bronchoscopes inserted into the airways, gastroscopes for investigation of the stomach, etc. Gastroscopes, for instance, are smaller in diameter than colonoscopes; bronchoscopes are smaller again and shorter in length while duodenoscopes have a different tip design to access the bile duct. Other types of endoscopes, such as a pleuroscope, an intubation scope, a laryngoscope, a rhino laryngoscope, a cystoscope, a ureteroscope, and a hysteroscope, further include different properties.

A variety of options are available to mechanically remove biological residues from the channel which is the first stage in the cleaning and disinfection process. One procedure for cleaning the channels utilizes small brushes mounted on long, thin, flexible lines. Brushing is the mandated means of cleaning the channel in some countries. These brushes are fed into the channels while the endoscope is submerged in warm water and a cleaning solution. The brushes are then pushed/pulled through the length of the channels in an effort to scrub off the soil/bio burden. Manual back and forth scrubbing is typically required. Water and cleaning solutions are then flushed down the channels. These flush-brush processes are repeated three times or until the endoscope reprocessing technician is satisfied that the channel is clean. At the end of this channel cleaning process air is pumped down the channels to dry them. A flexible pull-through device having wiping blades may also be used to physically remove material. A liquid flow through the channel at limited pressure can also be used.

In general, however, only the larger suction/biopsy channels (e.g., 122 in FIG. 1) can be cleaned by brushing or pull throughs. Air/water channels (e.g., channels 124 and 126) are generally too small for brushes so these channels are usually only flushed with water and cleaning solution.

After mechanical cleaning, a chemical cleaning may be conducted to remove the remaining biological contaminants. However, because endoscopes are sensitive and expensive medical instruments, the biological residues cannot be treated at high temperatures or with strong chemicals. For this reason, the mechanical cleaning needs to be as thorough as possible. In many cases, the current mechanical cleaning methodologies fail to fully remove biofilm from channels, particularly where cleaning relies on liquid flow alone. Regardless of how good the conventional channel cleaning processes are, it is almost inevitable that a small microbial load will remain in the channel of the lumen.

There has been significant research to show that the method of cleaning with brushes, even when performed as prescribed, may not completely remove biofilm in endoscope channels. As well as lacking in efficacy, the current manual brushing procedures suffer from other drawbacks. The large number of different endoscope manufacturers and models results in many minor variations of the manual cleaning procedure. This has led to confusion and ultimately poor compliance in channel cleaning processes.

The current system of manual brushing is also labor intensive, leading to increased cost. Thus, the current approaches to cleaning and disinfecting the channels in medical cleaning apparatus are still inadequate and residual microorganisms are now recognized as a significant threat to patients and staff exposed to these devices. For example, there is evidence of bacterial transmission between patients from inadequate cleaning and disinfection of internal structures of endoscopes which in turn has led to patients acquiring mortal infections. Between 2010 and 2015 more than 41 hospitals worldwide, most in the U.S., reported bacterial infections linked to the scopes, affecting 300 to 350 patients (http://www.modernhealthcare.com/article/20167415/NEWS/167419935). It would be expected that a reduction in the bioburden in various medical devices would produce a concomitant overall reduction in infection rates and mortality.

In addition, if endoscopes are not properly cleaned and dried, biofilm can build up on the channel wall. Biofilms start to form when a free-floating microorganism attaches itself to a surface and surrounds itself with a polysaccharide layer. The microorganism then multiplies, or begins to form aggregates with other microorganisms, increasing the extent of the polysaccharide layer. Multiple sites of attachment can in time join up, forming significant deposits of biofilm. Once bacteria or other microorganisms are incorporated in a biofilm, they become significantly more resistant to chemical and mechanical cleaning than they would be in their free-floating state. The organisms themselves are not inherently more resistant, rather, resistance is conferred by the polysaccharide film and the fact that microorganisms can be deeply embedded in the film and isolated from any chemical interaction. Any residual biofilm remaining after an attempt at cleaning quickly returns to an equilibrium state and further growth of microorganisms within the film continues. Endoscopes channels are particularly prone to biofilm formation. They are exposed to significant amounts of bioburden, and subsequent cleaning of the long narrow channels is quite difficult due to inaccessibility and the inability to monitor the channel cleaning process.

There is considerable pressure in medical facilities to reprocess endoscopes as quickly as possible. Because endoscopes are cleaned by hand, training and attitude of the technician are important in determining the cleanliness of the device. Residual biofilm on instruments can result in a patient acquiring an endoscope acquired infection. Typically, these infections occur as outbreaks and can have fatal consequences for patients.

Characterizing cleaning efficacy of an endoscope is also difficult. Indeed, the portions of lumens of an endoscope are difficult to access and inspect. For example, samples from an endoscope are extracted to measure cultivation of bacteria from the samples to gauge the effectiveness of the channel cleaning process. However, such extraction, cultivation, and measurement can be tedious, time consuming, and expensive. Moreover, manually monitoring a cleaning action can be complicated and would involve training an operator. Even upon successfully training an operator, human error during monitoring can lead to inaccuracies, resulting in ineffective cleaning.

Presented herein are techniques that overcome or ameliorate at least one of the disadvantages of the prior art, or to provide a useful alternative, through the use of a cleaning fluid to clean device channels. In particular, and as described in greater detail below, presented herein are techniques for monitoring, evaluating, analyzing, or otherwise characterizing a cleaning efficacy of a cleaning process using a cleaning fluid delivered to/through a device channel. More specifically, in accordance with embodiments presented herein, a cleaning fluid is delivered to (through) a channel (through the lumen defined by the channel). While the cleaning fluid passes through the channel, the cleaning efficacy of the channel (lumen) cleaning process is characterized (e.g., in terms of the current state of the channel cleaning process, an entirety of the channel cleaning process, only a portion of the channel cleaning process, etc.). In certain embodiments, a transducer (e.g., vibration sensor) is operably coupled to the channel and is configured to obtain a measurement of interaction (interaction measurement) between the cleaning fluid and the channel as the cleaning fluid passes there through. The interaction measurement can be, in turn, used to characterize a cleaning efficacy of the cleaning process. In certain examples, the interaction measurement may be a vibration measurement.

As noted, there are a number of different types of cleaning fluids that can be used to clean the channels of a device. These cleaning fluids include, for example, water, modified water, detergent solutions (e.g., mixtures of water and detergent), fluid-powder suspensions/liquid-powder mixtures (e.g., mixtures of sodium bicarbonate powder and water), etc. The use of a fluid-powder mixture propelled through respective channels of a device has been found to be particularly effective at removing unwanted matter via physical contact-yet safely-interacting with the channels to clean them. In these techniques, a fluid-powder mixture may be created, apportioned into a suitable amount, and then delivered at a suitable velocity through at least a portion of the channel. As referred to herein, a suitable velocity can refer to a velocity that can effectively move the fluid-powder mixture through the channel given the constraints (e.g., mechanical constraints, such as pressure ceiling) of the channel, as well as of the fluid-powder mixture. For example, a suitable velocity can be in the order of 1000 millimeters/second. The fluid-powder mixture is sometimes referred to herein as a ‘slurry,’ and the apportioned amount of the fluid-powder mixture is sometimes referred herein to as a ‘cleaning shot’ or ‘shot.’ Although certain aspects of the present disclosure are discussed with respect to usage of an apportioned amount of the fluid-powder mixture, the techniques described herein may be applied to any suitable flow, including a steady or continuous stream, of the fluid-powder mixture. Additionally, merely for ease of illustration, aspects of the techniques presented herein are described with reference to the use of fluid-powder mixtures. However, as noted elsewhere herein, the use of fluid-powder mixtures is merely illustrative and the techniques presented herein can be implemented with any cleaning fluid used in any of a number of different channel cleaning processes.

Referring specifically to fluid-powder mixtures, the fluid (e.g., liquid or gas) component of the fluid-powder mixture can facilitate the fluidity of the mixture, while the presence of the powder can physically interact with (i.e., clean) the interior surface/walls of the target channel to thereby remove contaminant adhering to the interior surface. In accordance with certain examples, the powder component of the fluid-powder mixture is present within the mixture in amounts greater than the respective saturation limit within the respective fluid, which can facilitate a cleaning interaction between the mixture and the walls of the channel. In certain embodiments, the fluid-powder mixture can comprise a mixture of silica powder and water. In other embodiments, the cleaning agent could comprise sodium chloride (NaCl), sodium bicarbonate (NaHCO₃), or other material. Where water soluble materials are used, they may be present in an amount greater than the respective saturation level. However, it is to be appreciated that any suitable fluid-powder mixture and suitable cleaning agent can be used in alternative examples. The specific properties of a given fluid-powder mixture can be selected/set in accordance with the specific properties of the channel to be cleaned. In some arrangements, the fluid-powder mixture is a liquid-powder suspension and the powder is present in an amount below the respective saturation of the associated liquid. However, the liquid is delivered to the target channel prior to the complete dissolution of the powder in the liquid. In this way, the undissolved powder can still interact with the target channel. Additionally or alternatively, as noted above, the techniques presented herein can use various fluid-powder mixtures that comprise gas-powder mixtures.

Moreover, it is to be appreciated that the fluid-powder mixture can be created/obtained in any of a variety of ways. For example, in certain embodiments, a powder is obtained from a cartridge or other consumable chamber/container, water is obtained from a tap, and these constituent components are mixed within a holding chamber (or within the consumable chamber itself) proximate (e.g., within days or weeks) to the time of cleaning. This approach may be advantageous insofar as powders, such as sodium bicarbonate, can be relatively stable and can have a long shelf life and suitable sources of fluid, such as water or air, are readily available. However, in other embodiments, the mixture may be obtained in an already mixed form.

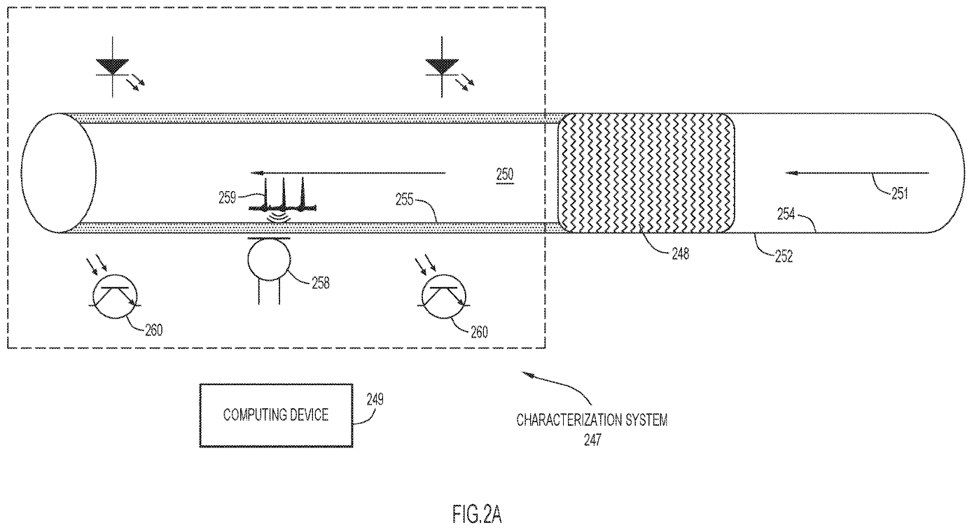

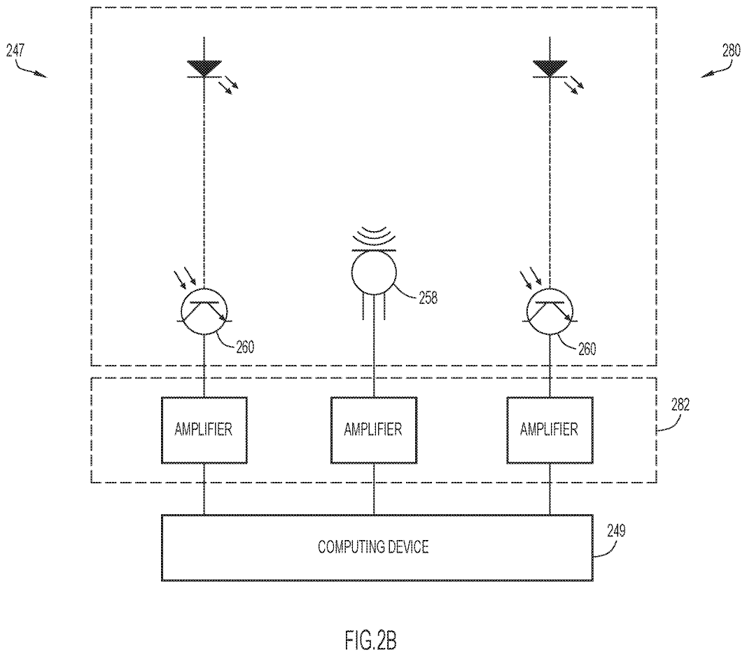

As noted, for ease of illustration and description, the techniques presented herein are primarily described with reference to cleaning channels with fluid-powder mixtures and, more specifically, with reference cleaning channels with apportioned amounts of fluid-powder mixtures (e.g., cleaning shots). FIG. 2A illustrates an embodiment in which a cleaning shot of a fluid-powder mixture is apportioned into a suitable amount before being delivered to a channel of a medical device channel during a monitoring process in accordance with embodiments presented herein.

In particular, FIG. 2A illustrates an embodiment in which a cleaning shot 248 (apportioned amount of a fluid-powder mixture) is delivered to a lumen 250 of a medical device channel 252 where the general direction of travel of the cleaning shot 248 is represented by arrow 251. As shown in FIG. 2A, the medical device channel 252 has an interior surface/wall 254 that has some amount of contaminant buildup 255 (e.g., bioburden, flesh, blood, mucus, feces, biofilm, lubricant). In operation, a plurality of cleaning shots 248 are separately delivered through the lumen 250 and the plurality of cleaning shots 248 is collectively configured to remove the contaminant buildup 255 from the interior surface 254 of the channel 252.

In general, cleaning shots 248 presented herein can have different forms/arrangements. For example, in certain embodiments, a cleaning shot 248 presented herein can be a relatively singular/unitary mass (e.g., potentially substantially occluding the lumen 250 of channel 252 while traveling therethrough), which is sometimes referred to herein as a “unitary shot.” However, in other embodiments, a cleaning shot 248 can be an “agglomeration” or “cluster” of smaller masses/groups that travel through the lumen 250 of channel 252 as a loose group (e.g., potentially not occluding the lumen 250 of channel 252 while traveling therethrough), sometimes referred to herein as a “cluster shot.” FIG. 2A schematically illustrates an example in which the cleaning shot 248 is a unitary shot.

In certain embodiments, a cleaning shot 248 can transition between different forms during its life cycle. For example, a cleaning shot 248 could be apportioned (initially created) as a unitary shot, but then transition to a cluster shot. This transition could occur before entering the channel 252 (e.g., in a delivery chamber) and/or while traveling through the channel 252.

In certain embodiments, a fluid flow (e.g., water) is interspersed between the delivery of cleaning shots 248. In other embodiments, multiple cleaning shots 248 could alternatively be delivered through the channel 252 either simultaneously or sequentially, without separation (e.g., without a fluid-only flow).

Importantly, it should be appreciated that in certain embodiments, the cleaning shots 248 are delivered through the channel 252 sequentially (e.g., one-at-a-time). In general, the use of a series of discrete/individual cleaning shots 248, as opposed to a single large flow, can allow the individual cleaning shots 248 to maintain sufficient kinetic energy to pass through the channel 252 at a rate that allows the particles within the cleaning shots 248 to advantageously interact with and clean the interior surface 254.

As noted above, in the embodiment of FIG. 2A, the fluid-powder mixture is apportioned into suitable amounts of cleaning shots 248 that are subsequently delivered through the target channel 252. Delivering discrete amounts of the fluid-powder mixture (cleaning shots 248) can be advantageous insofar as the discrete amounts can be delivered periodically with a certain set of attributes, such as at suitable speeds (e.g., around 1000 millimeters/second), with suitable pressures (e.g., between 60 and 250 kilopascals) in the lumen 250, having suitable volumes, and/or using suitable compositions (e.g., percentage composition of liquid, gas, and powder and/or percentage composition of various materials). Such attributes can be readily adjusted based on certain characteristics (e.g., a cross-sectional area) of the target channel 252. The periodic application of the fluid-powder mixture can help facilitate the cleaning of the channel 252 while not clogging/blocking the channels 252. Moreover, the discrete nature of the delivered amounts can facilitate the maintenance and/or adjustment of the attributes, which can also aid cleaning. For example, the use of cleaning shots 248 may reduce the risk of ‘clogging’ or otherwise obstructing the channel to reduce the velocity at which the fluid-powder mixture flows through the channel, while removing a desirable amount of contaminant from the interior surface 254.

Notably, different attributes of cleaning shots 248 may be suitable for the different characteristics of channels. For example, air/water channels within an endoscope are typically amongst the narrowest channels and, accordingly, may be more suitably cleaned with relatively smaller amounts of a fluid-powder mixture (e.g., cleaning shots 248 having a relatively smaller volume or clustering), whereas using larger amounts of a fluid-powder mixture (e.g., cleaning shots 248 having a relatively larger volume or clustering) may result in blocking such a narrow channel. In embodiments using a series of discrete cleaning shots 248, a relatively lower quantity of cleaning shots 248 (e.g., 3 cleaning shots 248) and/or a relatively greater time delay (e.g., 30 seconds) between cleaning shots 248 is utilized between the cleaning shots 248 to avoid blocking the narrow channel. In contrast, the suction/biopsy channels of an endoscope are typically amongst the widest channels and, accordingly, may be more suitably cleaned with relatively larger amounts of a fluid-powder mixture. For embodiments using a series of cleaning shots 248, a relatively greater quantity of cleaning shots 248 (e.g., 21 cleaning shots 248) and/or a relatively smaller time delay (e.g., 15 seconds) between cleaning shots 248 can be utilized without blocking the wide channel. As such, the amount of fluid-powder mixture apportioned for use in cleaning a given channel can be a function of the geometry of the channel. It should of course be appreciated that the amount of fluid-powder mixture apportioned can also or alternatively be a function of any of a variety of parameters.

An apportioned amount of the fluid-powder mixture can be established in any of a variety of ways. For example, in certain embodiments, a valve may be used to draw a target amount of fluid-powder mixture from a reservoir. In some embodiments, a self-regulating pressurized system is used to draw a suitable amount of fluid-powder mixture from the reservoir.

As noted, FIG. 2A illustrates the delivery of one cleaning shot 248 (e.g., an apportioned amount of a fluid-powder mixture) through the lumen 250 to clean the channel 252, where the general direction of travel of the cleaning shot 248 is represented by the arrow 251. The cleaning shot 248 can be considered to be entrained within a carrier fluid (e.g., air, water, etc.) used to deliver (e.g., push) the fluid-powder mixture through at least a portion of the channel 252 at a suitable velocity. In general, the fluid-powder mixture is delivered in a manner (e.g., suitable size, suitable velocity, etc.) to provide an appropriate physical interaction between the fluid-powder mixture and the interior surface 254 of the channel 252 to cause the undissolved powder to physically contact or run against the contaminant buildup 255 on the interior surface 254. In other words, the cleaning shot 248 is intended to physically interact with the interior surface 254 of the channel 252 and thereby abrade the contaminant buildup 255 on the interior surface 254 to remove (e.g., dislodge, scrape, detach) the contaminant buildup 255 from the interior surface 254.

As noted above, although the present disclosure primarily discusses utilizing the fluid-powder mixture as cleaning shots 248, it is to be appreciated that in additional or alternative embodiments, the fluid-powder mixture can be delivered through the lumen 250 as a continuous flow. A flow is referred to herein as being “continuous” in that a contiguous stream of the fluid-powder mixture is delivered to a lumen 250 over a period of time.

As noted above, a channel cleaning process, such as described above with reference to FIG. 2A, can be implemented in a number of different manners with a number of different channels. For context, one specific example implementation is described with reference to cleaning at least part of the endoscope 100 of FIG. 1.

More specifically, in one example channel cleaning process/cycle, one or more cleaning shots 248 are fired/shot into the water-jet channel 128 via the water-jet connector 139, one or more cleaning shots 248 are then fired into the biopsy/suction channel 122 via the suction connector 137, one or more cleaning shots 248 are then fired into the water-jet channel 128 via the water-jet connector 139, one or more cleaning shots 248 are then fired into the distal section 122B of the biopsy/suction channel 122 via the biopsy valve 118, one or more cleaning shots 248 are then fired into the water-jet channel 128 via the water-jet connector 139, and then one or more cleaning shots 248 are fired into the biopsy/suction channel 122 via the suction connector 137. The cleaning cycle can further include firing/shooting one or more cleaning shots 248 into the air channel 124 via the air connector 143, firing one or more cleaning shots 248 into the water channel 126 via the water connector 141 (e.g., in parallel). The firing of the cleaning shots 248 within each target channel can be preceded by, or followed by, a fluid flow. The cleaning shots 248 and fluid flows can be delivered via one or possible multiple connectors (e.g., one connector for the air pump 112 and one connector for the water bottle 110).

Returning to the example of FIG. 2A, as discussed above, the cleaning shots 248 are delivered to the channel 252 to remove the contaminant buildup 255 from the interior surface 254 of the channel 252. In accordance with embodiments presented herein, the channel cleaning process can be monitored to ensure that the attributes of the cleaning shots 248 are suitable/sufficient to remove the contaminant buildup 255 as desired/intended. That is, the cleaning efficacy of a channel cleaning process using the cleaning shots 248 can be characterized and, in certain examples, used to initiate one or more additional actions. For example, the cleaning efficacy of the cleaning shots 248 can be used to determine whether the contaminant buildup 255 has been desirably removed, the cleaning efficacy of the cleaning shots 248 be used to determine whether additional cleaning shots are needed, the cleaning efficacy of the cleaning shots 248 be used to determine whether attributes of any additional cleaning shots 248 should be adjusted, etc. Therefore, as noted above, reference to characterizing the cleaning efficacy of a channel cleaning process can refer to an analysis/evaluation/determination of the efficacy of the overall channel cleaning process and/or an efficacy of only portions of the channel cleaning process, such as the efficacy of a given cleaning fluid flow (e.g., the efficacy of one or more cleaning shots).

In accordance with embodiments presented herein, a cleaning efficacy characterization system 247 is provided. The cleaning efficacy characterization system 247 includes at least one transducer/sensor 258 operably coupled to the channel 252, and a computing device 249 is connected (e.g., via a wired or wireless connection) to the at least one transducer 258. The at least one transducer 258 is configured to measure interaction between the cleaning fluid and the channel 252 as the cleaning fluid passes through the channel 252. That is, the at least one transducer 258 is configured to capture “interaction measurements” that indicate or characterize the physical interaction between a cleaning shot 248 and the channel 252 as the cleaning shot 248 passes through the channel (e.g., as the cleaning shot travels through the lumen 250). However, although the present disclosure primarily discusses operation of the cleaning efficacy characterization system 247 to characterize cleaning efficacy of a channel cleaning process involving the use of the cleaning shot 248, the techniques discussed herein can be implemented to characterize any suitable channel cleaning process using a cleaning medium that interacts with the channel 252. For example, the cleaning efficacy characterization system 247 can characterize cleaning efficacy of a channel cleaning process in which a contiguous stream of fluid interacts with the channel 252 and/or in which an air or liquid (e.g., that does not contain powder) interacts with the channel 252. Furthermore, the techniques discussed herein regarding a cleaning fluid can also be applied to any other cleaning medium, including a cleaning medium that uses an abrasive solid (e.g., for a manual brushing or sandblasting process) that interacts with the channel.

In one example, the at least one transducer 258 is at least one vibration sensor 258 (e.g., representative of one or more vibration sensors), such as a piezoelectric sensor, is used to capture interaction measurements in the form of vibration measurements generated as a result of an interaction (e.g., a physical interaction, a mechanical interaction) between the cleaning shot 248 and the channel 252 (e.g., the interior surface 254 of the channel) during movement of the cleaning shot 248 through the lumen 250. By way of example, the vibration can indicate flow of the fluid-powder mixture against the interior surface 254 as caused by movement of the cleaning shot 248 through the lumen.

In other words, in this example, the at least one vibration sensor 258 captures/obtains one or more vibration measurements 259 representative of the interaction between the cleaning shot 248 and the interior surface 254. The vibration measurements 259 are analyzed at the computing device 249 to characterize a cleaning efficacy of a channel cleaning process provided by the cleaning shot 248. While not being bound by any specific theory, the vibration measurement can include sound waves and/or mechanical vibration of the channel 252. To this end, the vibration sensor 258 can include a vibration transducer, a microphone, an accelerometer, or any other suitable type of sensor configured to measure vibration. Sufficient cleaning efficacy may be particularly indicated by sufficiently turbulent flow of the fluid-powder mixture against the interior surface 254 to impart sufficient shear force to remove the contaminant buildup 255 from the interior surface 254. Vibration measurements 259 obtained by the vibration sensor 258 may indicate whether sufficiently turbulent flow of the fluid-powder mixture is generated by movement of the cleaning shot 248 through the channel to provide the desirable interaction between the cleaning shot 248 and the interior surface 254 for removing the contaminant buildup 255.

As further discussed herein, one or more attributes/properties of the vibration measurements generated by movement of the cleaning shot 248 through the lumen 250 can be analyzed (e.g., compared to one or more corresponding target values) to characterize whether the level of cleaning efficacy of the cleaning shot 248 is sufficient. For example, in response to characterizing that the level of cleaning efficacy of the cleaning shot 248 is insufficient based on the associated vibration measurements, one or more additional cleaning shots 248 can be delivered through the channel 252 to provide further cleaning of the interior surface 254.

In a further example, attributes/properties of the one or more additional cleaning shots 248 can be adjusted (e.g., based on the vibration measurements or other measurements, as described below) to change the physical interaction between the one or more additional cleaning shots 248 and the channel 252. For instance, as mentioned above, each cleaning shot 248 can be delivered with a certain set of attributes. Such attributes can also be adjusted between each cleaning shot 248 to provide a desirable level of cleaning efficacy. By way of example, in response to characterizing a level of cleaning efficacy provided by a first cleaning shot 248 delivered/provided with a first set of attributes is insufficient, a second cleaning shot 248 can be delivered through the channel 252 with a second set of attributes that are different from the first set of in an attempt to provide a sufficient level of cleaning efficacy.

In certain examples, additional sensors 260 of the cleaning efficacy characterization system 247 can also be utilized to facilitate monitoring of the cleaning of the interior surface 254, such as to determine the set of attributes used to deliver the cleaning shots 248. As an example, the additional sensors 260 can include optical sensors (e.g., a camera), position sensors, or any other suitable sensor configured to determine a position of the cleaning shot 248 within the channel 252 over time, which can indicate the speed of travel of the cleaning shot 248 through the channel 252, a cross-sectional area of the cleaning shot 248 (e.g., relative to a cross-sectional area of the lumen 250), and/or a volume of the cleaning shot 248 (e.g., derived based on the speed of travel of the cleaning shot 248, a length of the cleaning shot 248, and/or the cross-sectional area of the cleaning shot 248). To this end, the additional sensors 260 are offset from one another to provide spacing that enables detecting a positional change of the cleaning shots 248 for determining the movement of the cleaning shots 248 through the channel 252. For example, the additional sensors 260 are positioned at opposite sides of one of the transducers 258. In other words, one of the transducers 258 can be positioned between a pair of additional sensors 260 such that one additional sensor 260 is positioned upstream of the transducer 258 and another additional sensor 260 is positioned downstream of the transducer 258. However, in alternative embodiments, a single additional sensor 260 can be utilized. As another example, the additional sensors 260 can include a pressure sensor configured to determine a pressure within the lumen 250 during movement of the cleaning shot 248 through the channel 252. As a further example, the additional sensors 260 can measure an electrical property of the cleaning shot 248, which can be used to determine a composition (e.g., a water/bicarbonate ratio, liquid/powder ratio) of the cleaning shot 248. Further still, the additional sensors 260 can include a force sensor that directly measures shear force of the cleaning shot 248 imparted against the interior surface 254 can be used. In any case, the measurements provided by the additional sensors 260 can be used to determine the set of attributes of the cleaning shot 248 and for adjusting the set of attributes accordingly (e.g., in response to the level of cleaning efficacy provided by the cleaning shot 248 being insufficient). In certain embodiments, one of the additional sensors 260 can be positioned upstream of and adjacent to the transducer 258 to indicate the cleaning shot 248 will soon be able to detect an interaction between the cleaning fluid and the channel 252. That is, the additional sensor 260 detecting the presence of the cleaning shot 248 provides advance notice that the cleaning shot 248 is approaching the area being measured by the transducer 258.

Moreover, in some embodiments, the additional sensors 260 can be used to characterize cleaning efficacy over a duration of time. For instance, the additional sensors 260 monitor a particular time during which each cleaning shot 248 travels through the channel 252. Additionally, the transducer 258 determines the cleaning efficacy of each cleaning shot 248. Thus, the cleaning efficacy, as determined by the transducer 258, associated with each particular time of travel of the cleaning shots 248, as determined by the additional sensors 260, can be monitored. The cleaning efficacy over time can be presented as a timing diagram that indicates a trend of cleaning efficacy as cleaning shots 248 are delivered through the channel 252.

Although each of the sensors 258, 260 are positioned external to the lumen 250 in the illustrated embodiment, in certain embodiments, at least a portion of the sensors 258, 260 can be positioned within the lumen 250, such as against the interior surface 254, for determining the set of attributes of the cleaning shot 248. In embodiments in which the sensors 258, 260 are positioned external to the lumen 250, the channel 252 can have certain properties (e.g., a degree of transparency, a degree of translucency, a degree of electrical conductance) to enable the sensors 258, 260 to obtain the measurements indicating the set of attributes. In embodiments in which the sensors 258, 260 are positioned within the lumen 250, a cover, shield, or other protection can be positioned over the sensors 258, 260 to block contaminant buildup on the sensors 258, 260, to shield the sensors 258, 260 from the cleaning shot 248, or to otherwise protect the sensors 258, 260 from elements within the lumen 250.

FIG. 2B illustrates the cleaning efficacy characterization system 247 in greater detail. The transducer 258 and the additional sensors 260 of the cleaning efficacy characterization system 247 collectively form a sensor array/suite 280. Output of the sensor array 280 (e.g., of the transducer 258, of the additional sensors 260) is amplified by amplifiers 282 before being subsequently transmitted, such as to the computing device 249. The output of the sensor array 280 can additionally or alternatively be filtered to aid in analysis of signals that indicate mechanical interactions between the cleaning fluid and the lumen. In certain embodiments, the sensor array 280 includes an enclosure, such as tubing, to secure the transducer 258 and the additional sensors 260 to one another and to help couple the sensor array 280 to the channel. For instance, the sensor array 280 can be a standalone system implemented during a channel cleaning process for the channel 252. In some implementations, the sensor array 280 can also be used for monitoring cleaning efficacy of other types of channel cleaning processes, such as mechanical cleaning interactions (e.g., by brushing).

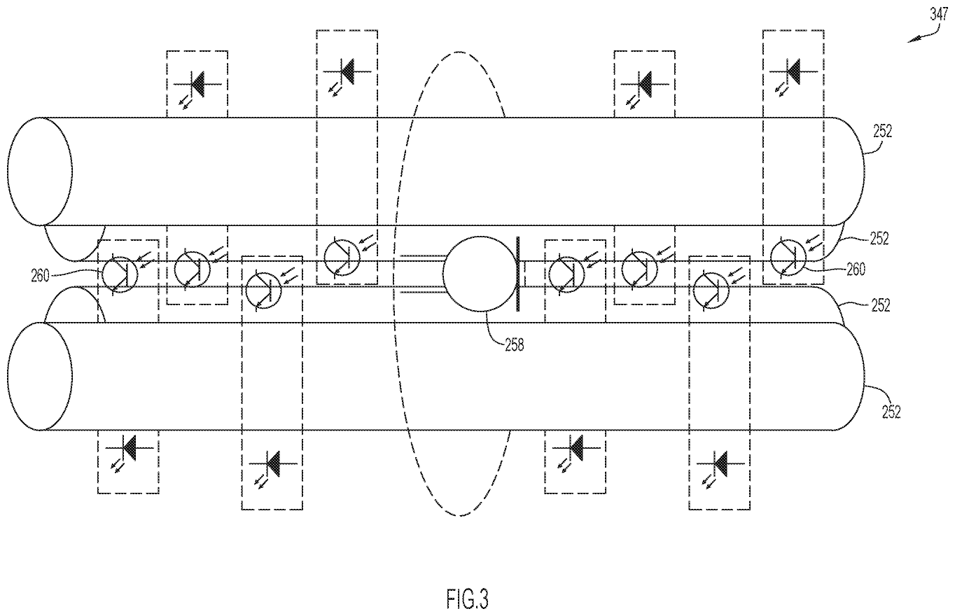

FIG. 3 is a schematic diagram of a cleaning efficacy characterization system, referred to cleaning efficacy characterization system 347, in which the system includes a single vibration sensor 258 configured to obtain vibration measurements associated with multiple different channels 252 (e.g., of the same endoscope). For example, the cleaning efficacy characterization system 347 is arranged such that the vibration sensor 258 is attached to or contacts each channel 252 (e.g., by surrounding the channels 252 around the vibration sensor 258, by surrounding the channels 252 with the vibration sensor 258) to obtain the vibration measurements generated as a result of respective cleaning shots 248 delivered through the channels 252. In certain embodiments, each channel 252 is individually cleaned. That is, the respective cleaning shots 248 are delivered to each channel 252 at a different period of time. For this reason, the vibration measurement obtained by the vibration sensor 258 at a certain time can be associated with a particular channel 252 based on the period of time in which the corresponding cleaning shot 248 is delivered through the particular channel 252. Such a configuration can ease the process of cleaning multiple channels 252 and characterizing the provided cleaning efficacies of the channels 252. For instance, multiple channels 252 can be effectively cleaned without having to perform separate cleaning operations that each utilizes a dedicated (e.g., separate) vibration sensor 258. As such, a time and/or cost associated with cleaning the channels 252 can be reduced.

Moreover, in some embodiments, each individual additional sensor 260 can be used to obtain measurements of respective cleaning shots 248 delivered through different channels 252. That is, one additional sensor 260 can help determine attributes of cleaning shots 248 delivered through a first channel 252 and attributes of cleaning shots 248 delivered through a second channel 252, different from the first channel 252. In this manner, usage of dedicated, additional sensors 260 that help determine attributes of cleaning shots 248 delivered through a single channel 252, and no other channels 252, can be avoided, thereby reducing a quantity of additional sensors 260 to be implemented. Thus, a cost associated with cleaning the channels 252 can be further reduced.

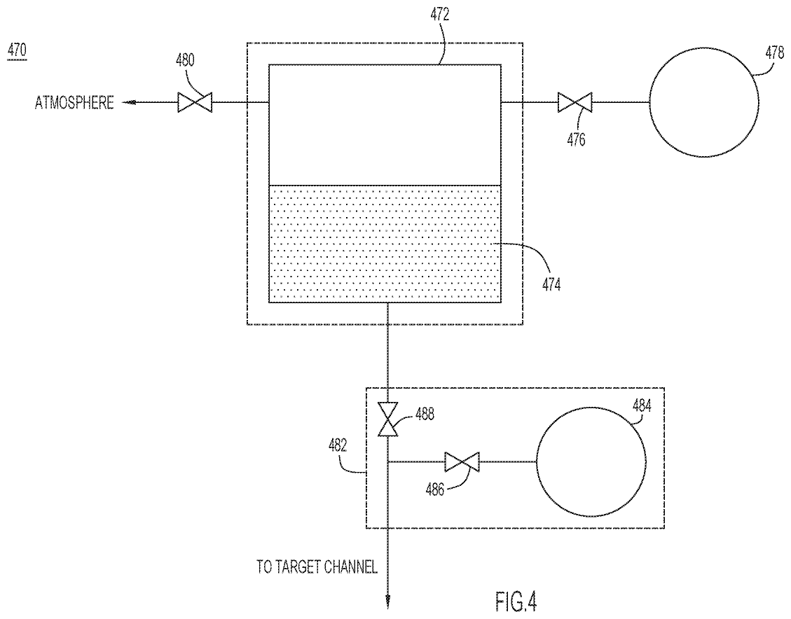

It is to be appreciated that systems for cleaning a channel of a medical device, in accordance with embodiments presented herein, can take any of a number of different forms/arrangements. In many embodiments, the systems can include: a holding chamber for creating/housing a powder and/or fluid-powder mixture and a mechanism for delivering a portion of the fluid-powder mixture to the target channel. FIGS. 4-7 illustrate various aspects of example systems that can be implemented in accordance with embodiments presented herein.

FIG. 4 illustrates a system 470 for cleaning a channel of a medical device using a fluid-powder mixture, in accordance with embodiments presented herein. More specifically, the system 470 includes a holding chamber 472 for creating/housing/retaining a fluid-powder mixture 474. In the illustrated embodiment, the holding chamber 472 is provided with the powder that is used to form the fluid-powder mixture 474. For example, the holding chamber 472 may be a consumable component of the system 470 and may be replaced when its contents have been used. In this context, ‘consumable’ can be understood as not being intended to be permanent fixtures of the systems with which they interact. For instance, a consumable holding chamber can be obtained, made to interface with a respective cleaning system, and once its components within it have been used up by the cleaning system, the consumable holding chamber may be disposed of or else sent to a center for reprocessing. Subsequently, a user can obtain another consumable holding chamber to perform further cleaning. The use of such consumable holding chambers can greatly enhance the efficiency and operability of the disclosed cleaning systems.

The holding chamber 472 interfaces with an inlet valve 476 for receiving liquid from a liquid source 478. A relief valve 480 can be used to relieve pressure created during creation of the fluid-powder mixture 474. As can be appreciated, the fluid-powder mixture 474 can be created using any suitable constituent components. For example, in certain embodiments, the powder that is provided with the holding chamber 472 is sodium bicarbonate, and the liquid source 478 is a source for water. Of course, it can be appreciated that the holding chamber 472 can receive liquid and powder in any of a variety of ways in accordance with embodiments of the disclosure. For example, in some embodiments, the chamber is configured to receive powder from a powder reservoir, e.g., a sodium bicarbonate cartridge. As alluded to above, the composition of the fluid-powder mixture 474 and the parameters for its delivery can be specifically selected to manifest the cleaning action. In some embodiments, a pump is used to provide liquid to the chamber in lieu of directly using a valve to do so. In some embodiments, the holding chamber 472 may include mechanisms (not illustrated) for facilitating the mixing of the received powder and liquid. For example, a stirring mechanism or an agitation mechanism may be implemented to facilitate mixing.

The system 470 further includes a delivery mechanism 482 for delivering a portion of the fluid-powder mixture 474 to the target channel. In the illustrated embodiment, the delivery mechanism is in the form of an aggregate of a carrier fluid source 484, a first valve 486, and a second valve 488. As can be appreciated, the carrier fluid may be made to flow through the target channel via the first valve 486, and portions of the fluid-powder mixture 474 may be entrained within this flow. It should be noted that any suitable carrier fluid may be implemented. For example, the carrier fluid may comprise at least one of: air, water, ethanol, nitrogen, and carbon dioxide. In the illustrated embodiment, the second valve 488 is configured to implement the amount of fluid-powder mixture 474 that is entrained within the carrier fluid. For example, the second valve 488 may be open to the holding chamber 472, and the holding chamber 472 may be pressurized via the liquid source 478 and the inlet valve 476, thereby resulting in delivery of a portion of the fluid-powder mixture 474 to the delivery mechanism 482. Of course, it should be appreciated that any suitable mechanism for implementing the amount to be entrained within the carrier fluid may be applied in accordance with embodiments of the present disclosure.

While one system architecture for cleaning a medical device having a channel has been illustrated, it should be appreciated that the described concepts can be implemented in any of a variety of ways in accordance with embodiments of the disclosure. For example, in some embodiments, the carrier fluid source 484 is additionally used to create the fluid-powder mixture 474 and, as such, a separate liquid source (e.g., the liquid source 478) may not be necessary. In some embodiments, a selectable plurality of carrier fluid sources can be implemented. Thus, for instance, in some embodiments, a source of air and a source of water can each supply carrier fluid for delivery of fluid-powder mixture 474 to the channel, and the water source may further be used to facilitate the creation of the fluid-powder mixture 474.

In some embodiments, the holding chamber 472 may include a discrete pressure source to facilitate the delivery of a portion of the fluid-powder mixture 474 to the delivery mechanism 482, such that the liquid source 478 does not have to facilitate said delivery.

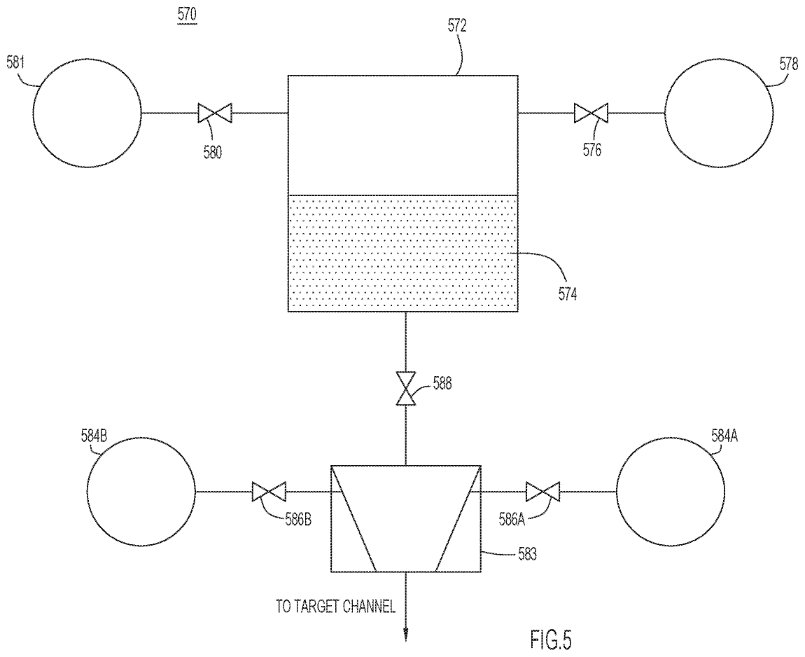

In certain embodiments, a separate chamber is implemented to facilitate the propulsion of the mixture through the target channel. For example, FIG. 5 illustrates a system 570 that includes a holding chamber 572 for creation/housing of a fluid-powder mixture 574 and a delivery chamber 583 for developing the velocity of the fluid-powder mixture 574 for subsequent delivery through the target channel. In the illustrated embodiment, a powder source 581 is coupled to the holding chamber 572 via a valve 580, and a liquid source 578 is coupled to the holding chamber 572 via a valve 576. The powder source 581 may be, for example, a cartridge and the liquid source 578 may be, for example, a pressure-regulated mains water.

As noted, the system 570 also comprises the delivery chamber 583 for delivery of a portion of the fluid-powder mixture 574 to the target channel. As shown, the delivery chamber 583 is coupled to each of two carrier fluid sources 584A and 584B via respective valves 586A and 586B. For example, air and water may serve as carrier fluids for the illustrated system 570. The amount of fluid-powder mixture 574 to be entrained in the carrier fluid can be controlled by a valve 588.

In general, one example purpose of a delivery chamber presented herein, such as the delivery chamber 583, is to create an air gap between the shot source (holding chamber 572) and the target channel. The delivery chamber 583 provides a region where the system 570 uses one or more fluids (e.g., air and/or water) to push the cleaning shots through the channel.

As shown in FIG. 5, the delivery chamber 583 defines a frustoconical shape, which can be beneficial in a number of respects. For example, such a geometry can aid the flow of the fluid-powder mixture, e.g., directing it towards the target channel. Additionally, the frustoconical shape may cause the development of a “vortex” of the carrier fluid within the delivery chamber 583.

It can be appreciated that while a certain configuration has been illustrated, systems implementing a discrete delivery chamber can be implemented in any of a variety of ways in accordance with embodiments of the disclosure. For example, in some embodiments, the delivery chamber is coupled to only a single carrier fluid source.

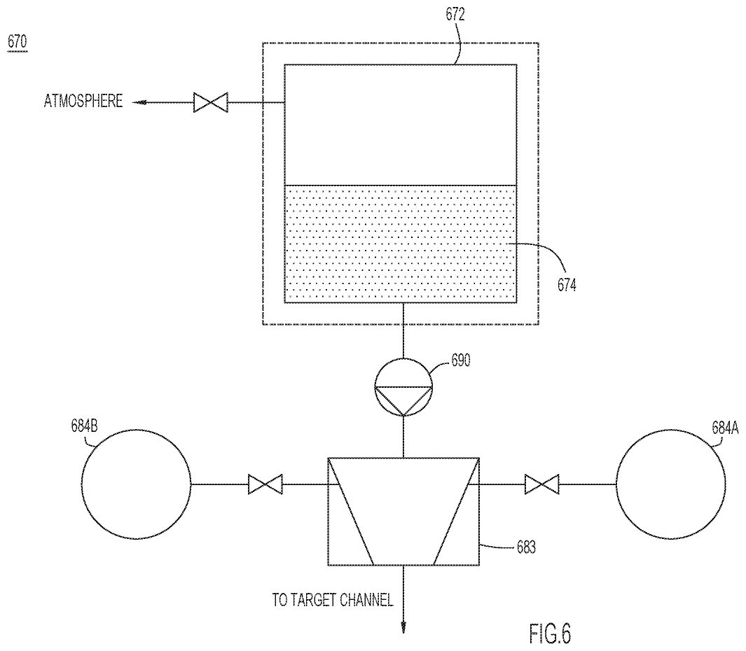

While the embodiment illustrated in FIG. 5 depicts an architecture whereby powder may be provided to a chamber via, for example, a cartridge, in some embodiments, the chamber may be a consumable component, as mentioned previously. Accordingly, FIG. 6 illustrates a system 670 for cleaning a channel of a medical device with a consumable component and a delivery chamber.

In particular, the system 670 includes a holding chamber 672 in the form of a consumable component that is provided with powder. A fluid-powder mixture 674 can be created/housed within the holding chamber 672 using liquid from carrier fluid source 684A. The system 670 further includes a carrier fluid source 684B that may house a gaseous carrier fluid. Similar to the system 570 of FIG. 5, the system 670 further includes a delivery chamber 683 operable to deliver the fluid-powder mixture 674 to the channel for cleaning. In certain embodiments, a pump 690 is also provided between the holding chamber 672 and the delivery chamber 683.

The use of a chamber in the form of a consumable component, as shown in FIG. 6, may be advantageous insofar as it simplifies the design and enhances user-operability. For example, the use of such a configuration can eliminate the need for a discrete powder handling mechanism. Although the illustrated embodiments depict a chamber that houses powder that is subsequently hydrated, in some embodiments, a chamber is provided with a pre-made fluid-powder mixture. For example, a chamber that houses a powder that is insoluble in a corresponding liquid may be implemented. The insolubility of the powder in the respective liquid may allow the chamber to have a suitable shelf life, and therefore may be commercially viable.

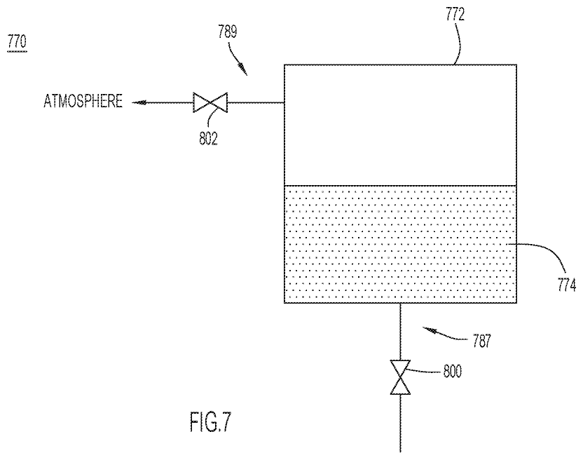

In certain embodiments, consumable chambers that house constituent components used for cleaning medical devices having lumens (e.g., those described elsewhere herein) are implemented. Accordingly, FIG. 7 illustrates a system 770 with a consumable holding chamber 772 housing at least one constituent component for use in a system for cleaning a lumen of a medical device. In particular, it is illustrated that the consumable holding chamber 772 includes a fluid-powder mixture 774 for use in a cleaning system (e.g., such as any of those described above). For example, in certain embodiments, the consumable holding chamber 772 is provided with sodium bicarbonate, and the cleaning system that interfaces with the consumable holding chamber 772 may thereafter provide the consumable holding chamber 772 with water to create a sodium bicarbonate-water mixture that can be used for cleaning. In some embodiments, the consumable holding chamber 772 inherently includes a fluid-powder mixture (e.g., prior to interfacing with a respective cleaning system). For example, certain fluid-powder mixture combinations may have a more durable shelf life (e.g., as compared to the mixture of sodium bicarbonate and water), and therefore, it may be more commercially viable for consumable chambers to include such fluid-water mixtures. It is further illustrated that the consumable holding chamber 772 includes two interfaces 787, 789 for engaging with a cleaning system. For instance, the interface 787 can allow the cleaning system to provide liquid to the consumable holding chamber 772 via a valve 800 to create the fluid-powder mixture 774 used for cleaning. The interface 787 may additionally/alternatively allow the cleaning system to draw a portion of the fluid-powder mixture from the consumable holding chamber 772 for subsequent delivery to a target lumen. The illustrated consumable holding chamber 772 further includes the interface 789 for engaging with a cleaning system. In particular, it is illustrated that the interface 789 allows the consumable holding chamber 772 to interface with a relief valve 802, which can help direct the fluid flow to/from the consumable holding chamber 772. In some embodiments, a third interface is included for engagement with a dedicated liquid source (e.g., similar to the arrangement shown in FIG. 4) for creating the fluid-powder mixture.

It should be appreciated that while specific configurations for a holding chamber have been illustrated, embodiments of the disclosure can be implemented in any of a variety of ways. As an example, a system presented herein further includes at least one distribution manifold that may couple to a plurality of ports/channels/lumens of the medical device to be cleaned. In several embodiments, a single delivery chamber is coupled to a single port of a medical device. Each of multiple delivery chambers may be simultaneously coupled to discrete ports of a single medical device. As another example, a system may include a holding chamber and a plurality of delivery chambers, and the holding chamber may provide cleaning shots to each of the plurality of the delivery chambers. In general, the disclosed concepts can be implemented in any of a variety of ways in accordance with embodiments of the disclosure.

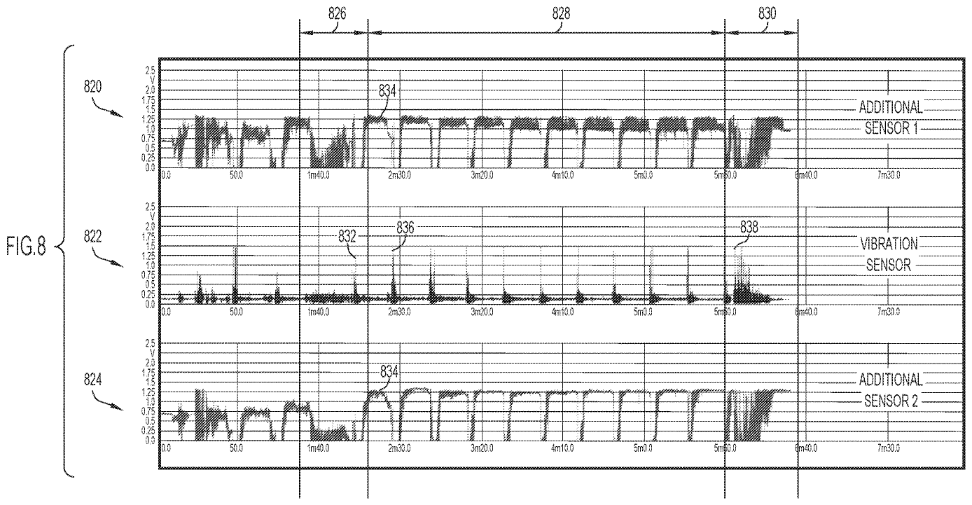

It is to be appreciated that the above-described concepts can be applied in any of a variety of ways in accordance with embodiments of the disclosure. Regardless of the manner in which a fluid-powder mixture is delivered to a channel (e.g., in the form of a cleaning shot), various parameters of the fluid-powder mixture can be determined during a channel cleaning process. FIG. 8 is a series of graphs illustrating of parameters captured by different sensors during a channel cleaning process in accordance with certain embodiments presented.

In particular, shown in FIG. 8 is: a first graph 820 illustrating measurements captured by a first additional sensor (e.g., an upstream additional sensor) over a duration of time of the channel cleaning process; a second graph 822 illustrating measurements captured by a vibration sensor (e.g., positioned downstream of the first additional sensor) over the duration of time; and a third graph 824 illustrating measurements captured by a second additional sensor (e.g., a downstream additional sensor positioned downstream of the vibration sensor) over the duration of time.

Different portions of each graph 820, 822, 824 correspond to different phases of the channel cleaning process. By way of example, a first portion 826 of each graph 820, 822, 824 corresponds to a pre-flush phase during which a liquid (e.g., water) is preliminarily delivered through the channel, a second portion 828 of each graph 820, 822, 824 corresponds to a cleaning phase during which a cleaning fluid is delivered through the channel, and a third portion 830 of each graph 820, 822, 824 corresponds to a post-flush phase during which a liquid (e.g., water) is delivered through the channel again.

At the first portion 826 of the first graph 820, the measurements of the first additional sensor include a relatively constant positive reading to indicate a continuous (e.g., steady) stream of liquid is delivered through the channel. Similarly, at the first portion 826 of the third graph 824, the measurements of the second additional sensor also include a relatively constant positive reading. At the first portion 826 of the second graph 822, the measurements of the vibration sensor include a peak amplitude 832 that indicates some interaction between the liquid and the channel (e.g., an interior surface).

At the second portion 828 of the first graph 820, the measurements of the first additional sensor include intermittent intervals of positive readings 834 that are each spaced apart by intervals during which signals of relatively low amplitude (e.g., below a threshold) are acquired. Each positive reading 834 corresponds to movement of a respective cleaning shot through the channel, and the signals of relatively low amplitude between each positive reading 834 corresponds to a delay between delivery of cleaning shots. At the second portion 828 of the third graph 824, the measurements of the second additional sensor include intermittent intervals of positive readings 834 that are each spaced apart by signals of relatively low amplitude. At the second portion 828 of the second graph 822, the measurements of the vibration sensor include peak amplitudes 836 that each indicate interaction between a respective cleaning shot and the channel. One way that efficacious cleaning can be indicated is if the peak amplitude 836 for a cleaning shot is greater than a threshold to indicate the cleaning shot is providing sufficient interaction to remove contaminant from the channel.

At the third portion 830 of the first graph 820, the measurements of the first additional sensor include another relatively constant positive reading to indicate a continuous (e.g., steady) stream of liquid is delivered through the channel. At the third portion 830 of the third graph 824, the measurements of the second additional sensor also include another relatively constant positive reading. At the third portion 830 of the second graph 822, the measurements of the vibration sensor include decreasing amplitudes 838, which may indicate remnants of loose contaminants and/or cleaning slugs being iteratively removed from the channel by the liquid.

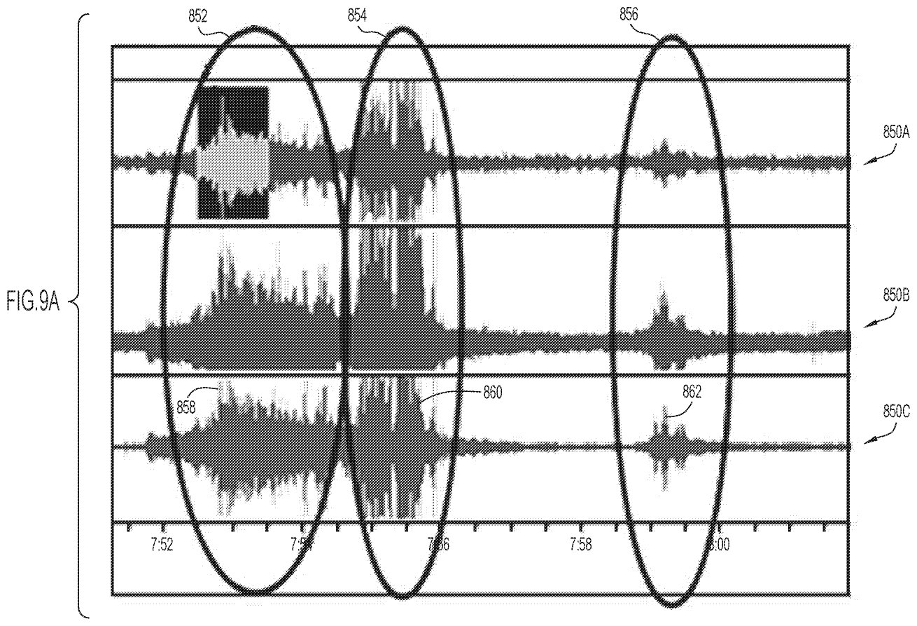

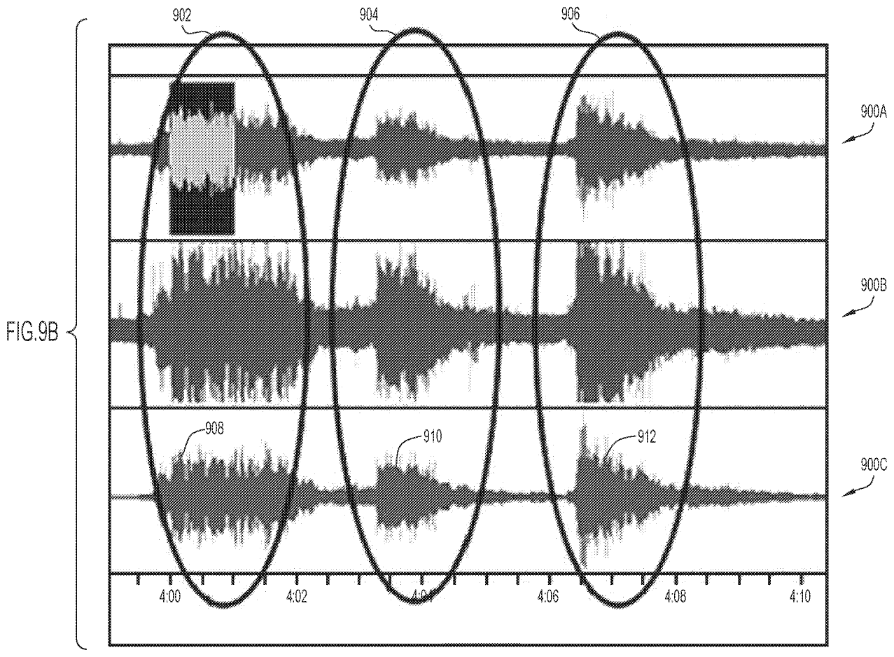

FIGS. 9A and 9B illustrate example vibration measurements obtained by a vibration sensor over a period of time (i.e., vibration measurements in the time domain). More specifically, FIG. 9A illustrates vibration measurements 850 obtained by a vibration sensor and indicating a sufficient level of cleaning efficacy provided by a cleaning fluid to a lumen of a channel of a medical device. Vibration measurements 850A include raw signals obtained by the vibration sensor, vibration measurements 850B include signals obtained after a low pass filter is applied to the vibration measurements 850A to remove signals above a threshold frequency (e.g., 15 kilohertz (kHz)), and vibration measurements 850C include signals obtained after a high pass filter is applied to the vibration measurements 850A to remove signals below a threshold frequency (e.g., 1200 Hz). The low pass filter and the high pass filter operate to aid in analysis of signals that indicate mechanical interactions between the cleaning fluid and the lumen. Each of the vibration measurements 850 includes a first portion 852, a second portion 854, and a third portion 856. The first portion 852 indicates vibration measurements generated via a cleaning shot delivered through the lumen, the second portion 854 indicates vibration measurements generated via an initial water flow delivered through the lumen subsequent to the cleaning shot, and the third portion 856 indicates a subsequent water flow delivered through the lumen subsequent to the initial water flow.

The first portion 852 of the vibration measurements 850C includes a peak amplitude 858, which is greater than a threshold value to indicate an interaction between the cleaning shot and the interior surface of the lumen sufficiently removed contaminant buildup from the interior surface. By way of example, the peak amplitude 858 exceeding the threshold value can indicate that the flow of fluid-powder mixture against the interior surface, as provided by the cleaning shot, sufficiently separates the contaminant buildup from the interior surface. The second portion 854 of the vibration measurements 850C includes amplitudes 860 that are also greater than the threshold value. For instance, after the cleaning shot has separated the contaminant buildup from the interior surface, the initial water flow delivered through the lumen moves the separated contaminant buildup, as well as remnants of the cleaning shot, along the interior surface for removal from the lumen. Movement of the contaminant buildup along the interior surface can cause the elevated amplitudes 860 (e.g., by impinging the contaminant buildup against the interior surface while moving the contaminant buildup along the interior surface). The third portion 856 includes amplitudes 862 that are below the threshold value. For example, the initial water flow has substantially removed the contaminant buildup from the lumen. Therefore, there may not be substantial amount of contaminant buildup remaining within the lumen to be moved by the subsequent water flow. As such, there may not be substantial interaction between the subsequent water flow and the interior surface. Thus, the vibration measurements at the third portion 856 are primarily generated by flow of the subsequent water flow along the interior surface, rather than flow of the subsequent water flow to move or detach contaminant buildup with respect to the interior surface.

FIG. 9B illustrates vibration measurements 900 obtained by a vibration sensor and indicating an insufficient level of cleaning efficacy provided by a cleaning fluid (e.g., water only at a low flow rate and without a cleaning mixture) to a lumen of a channel of a medical device. For example, the vibration measurements 900 are obtained when water is delivered through the lumen. Vibration measurements 900A include raw signals obtained by the vibration sensor, vibration measurements 900B include signals obtained after a low pass filter is applied to the vibration measurements 900B, and vibration measurements 900C include signals obtained after a high pass filter is applied to the vibration measurements 900A. The vibration measurements 900 include a first portion 902, a second portion 904, and a third portion 906. The first portion 902 indicates vibration measurements generated via a cleaning shot delivered through the lumen, the second portion 904 indicates vibration measurements generated via an initial water flow delivered through the lumen subsequent to the cleaning shot, and the third portion 906 indicates a subsequent water flow delivered through the lumen subsequent to the initial water flow.

The first portion 902 of the vibration measurements 900C includes a peak amplitude 908 that is less than a threshold value, which can indicate an interaction between the cleaning shot and the interior surface of the lumen did not sufficiently remove contaminant buildup from the interior surface. For example, the peak amplitude 908 being below the threshold value can indicate the flow of fluid-powder mixture against the interior surface, as provided by the cleaning shot, did not sufficiently separate the contaminant buildup from the interior surface. Consequently, a substantial amount of contaminant buildup remains attached to the interior surface. The second portion 904 of the vibration measurements 900C includes amplitudes 910 that are also less than the threshold value. As an example, because a sufficient amount of contaminant buildup remains attached to the interior surface, the initial water flow delivered through the lumen may not be able to move the contaminant buildup along the interior surface and therefore does not cause elevated amplitudes (e.g., otherwise caused by impingement of contaminant buildup against the interior surface during movement). The third portion 906 of the vibration measurements 900C includes amplitudes 912 that are also below the threshold value. Indeed, the subsequent water flow also may not be able to move a sufficient amount of the contaminant buildup along the interior surface and therefore does not cause elevated amplitudes. For this reason, each of the amplitudes 908, 910, 912 being below the threshold value may indicate an undesirable amount of contaminant buildup remains attached to the interior surface.

As noted, FIGS. 9A and 9B have been described with respect to comparing amplitudes (e.g., the amplitudes 860, the amplitudes 910) to a threshold value to characterize a cleaning efficacy. That is, in these examples, the threshold value has been established as representing a “signature” of an effective channel cleaning process (or effective cleaning fluid flow), sometimes referred to herein as “an effective channel cleaning process signature,” and the amplitudes are compared to the threshold value to determine whether received vibration measurements indicate a corresponding effective channel cleaning process. It is to be appreciated that in additional or alternative embodiments, other characteristics of vibration measurements may represent an effective channel cleaning process signature used to characterize cleaning efficacy. For example, in one alternative embodiment, a shape of the plot/curve of the vibration measurements in the time domain is compared to an expected shape indicative of a sufficient cleaning efficacy, meaning the expected shape represents an effective channel cleaning process (e.g., the shape matching the expected shape may indicate the channel cleaning process providing the vibration measurements is sufficiently effective). In certain examples, the characteristics (e.g., the threshold value, the expected shape, etc.) representing an effective channel cleaning process signature can be determined/generated through the use of artificial intelligence or other technique.

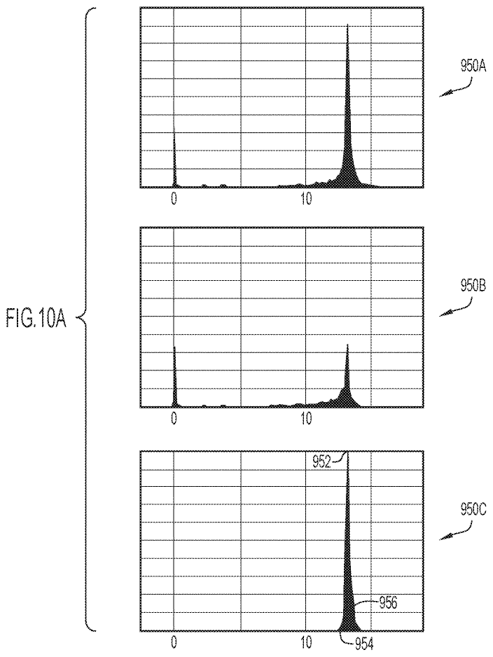

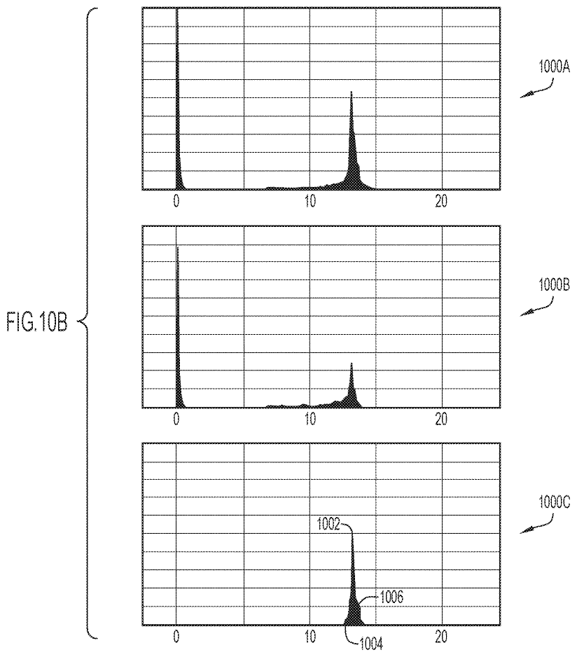

As noted, the examples of FIGS. 9A and 9B are illustrative and the cleaning efficacy of the channel cleaning process (e.g., the cleaning efficacy provided by one or more cleaning shots) can also or alternatively be characterized in another manner based on obtained vibration measurements. FIGS. 10A and 10B illustrate example quantities of vibration measurements, in the frequency domain, having certain frequencies (the quantity of obtained vibration measurements of each particular frequency). That is, each obtained vibration measurement includes a particular frequency, and some of the vibration measurements are of the same frequency.

In the examples of FIGS. 10A and 10B, vibration measurements with a greater frequency have been correlated with a more beneficial contaminant detaching result for increased cleaning efficacy. For example, such signals can indicate beneficial or sufficiently turbulent flow that can provide increased contaminant detachment. Therefore, a greater quantity of vibration measurements at higher frequencies can indicate greater cleaning efficacy. In certain embodiments, data associating the quantities of vibration measurements with different frequencies is provided by modifying (e.g., performing a Fast Fourier Transform of a time window of) data associating vibration amplitudes with time (e.g., as illustrated in FIGS. 9A and 9B).

Referring first to FIG. 10A, shown are quantities of vibration measurements 950 obtained by a vibration sensor and indicating a sufficient level of cleaning efficacy provided by a cleaning fluid to a lumen of a channel of a medical device. Vibration measurements 950A include quantities of raw signals at each frequency, vibration measurements 950B include quantities of signals at each frequency after a low pass filter is applied to the vibration measurements 950A to remove signals above a threshold frequency, and vibration measurements 950C include signals at each frequency after a high pass filter is applied to the vibration measurements 950A to remove signals below a threshold frequency.

As shown in FIG. 10A, a quantity 952 of vibration measurements 950C at or above a threshold frequency 954 after the filters are applied is greater than a threshold value. The quantity 952 exceeding the threshold value (e.g., to match an effective channel cleaning process signature) can indicate that there is a sufficient amount of interaction between the cleaning shot and the interior surface for removing contaminant buildup from the interior surface. Additionally or alternatively, a shape of a curve 956 plotting the quantity 952 of vibration measurements 950C at or above the threshold frequency 954 matching an expected shape corresponding to a sufficient level of cleaning efficacy can indicate an effective channel cleaning process (e.g., indicate there is a sufficient amount of interaction between the cleaning shot and the interior surface for removing contaminant buildup from the interior surface). In certain examples, an area under the curve 956 matching an expected area can indicate an effective channel cleaning process. The expected shape, expected area under a curve, or other effective channel cleaning process signature can be determined/generated via artificial intelligence or other technique.

FIG. 10B illustrates quantities of vibration measurements 1000 obtained by a vibration sensor and indicating an insufficient level of cleaning efficacy provided by a cleaning fluid to a lumen of a channel of a medical device. For instance, the vibration measurements 1000 are obtained when water (e.g., water and no other cleaning fluid flow) is delivered through the lumen. Vibration measurements 1000A include quantities of raw signals at each frequency, vibration measurements 1000B include quantities of signals at each frequency after a low pass filter is applied to the vibration measurements 1000A to remove signals above a threshold frequency, and vibration measurements 1000C include signals at each frequency after a high pass filter is applied to the vibration measurements 1000A to remove signals below a threshold frequency.

A quantity 1002 of vibration measurements 1000C at a threshold frequency 1004 after the filters are applied is less than a threshold value. The quantity 1002 being below the threshold value can indicate there is not a sufficient amount of interaction between the cleaning shot and the interior surface for removing contaminant buildup. Additionally or alternatively, a shape of a curve 1006 plotting the quantity 1002 of vibration measurements 1000C at or above the threshold frequency 1004 being different from an expected shape corresponding to a sufficient level of cleaning efficacy (e.g., different from an effective channel cleaning process signature) can indicate there is not a sufficient amount of interaction between the cleaning shot and the interior surface for removing contaminant buildup from the interior surface. For instance, an area under the curve 1006 being different from an expected area can indicate that there is not a sufficient level of cleaning efficacy.

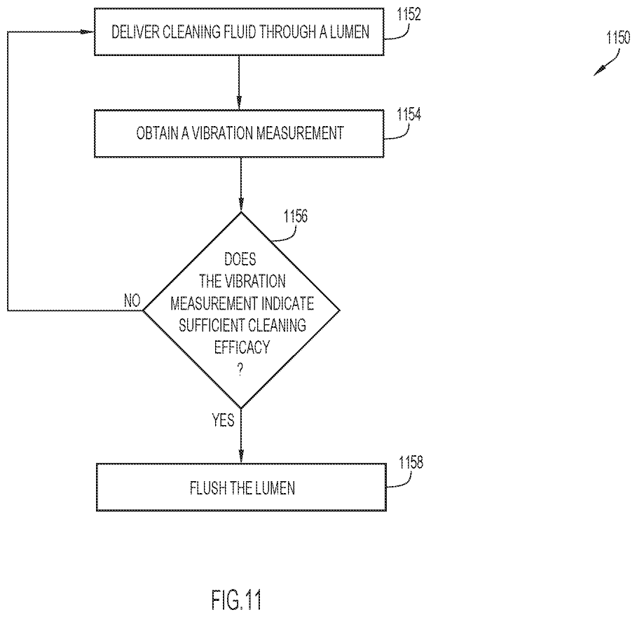

Each of FIGS. 11-13 discussed below illustrates a respective method related to cleaning a channel, such as of a medical device. In some embodiments, each method is performed by a common entity, such as the same computing device/system. In additional or alternative embodiments, different entities can perform operations of different methods. It should also be noted that each method can be performed differently than depicted. For example, an additional operation can be performed, a depicted operation can be performed differently, operations can be performed in different orders, and/or an operation may not be performed. Moreover, the respective operations of the different methods can be performed in any suitable order with respect to one another, such as sequentially and/or simultaneously.

FIG. 11 is a flowchart of a method 1150 for cleaning a channel of a medical device. At block 1152, a cleaning fluid (e.g., a first cleaning fluid flow), such as a cleaning shot of a fluid-powder mixture and/or a liquid (e.g., water), is delivered through a lumen of the channel. In certain embodiments, prior to delivering the cleaning fluid through the lumen, the lumen is preliminarily flushed with a liquid (e.g., water). For example, the liquid can wet the interior surfaces of the lumen and/or remove any initially loose debris within the lumen to facilitate subsequent travel of the cleaning fluid through the lumen. At block 1154, a vibration measurement is obtained while the cleaning fluid passes through the lumen. The vibration measurement, such as a sound measurement and/or a mechanical vibration measurement, is generated by an interaction between the cleaning fluid and an interior surface of the channel. In particular, the vibration measurement denotes whether such interaction indicates the cleaning fluid flows against the interior surface to remove contaminant buildup from the interior surface. As such, the vibration measurement is indicative of a physical interaction and/or mechanical interaction between the cleaning fluid and the lumen. In certain embodiments, the vibration measurement is filtered (e.g., by frequency) to provide signals that more accurately represent the interaction between the cleaning fluid and the interior surface, such as by removing noise or other signals that indicate negligible amounts of interaction between the cleaning fluid and the interior surface.

The cleaning efficacy of the cleaning process is then characterized (e.g., determined to be sufficient or insufficient) based on the vibration measurement. In particular, at block 1156, a determination is made regarding whether the vibration measurement indicates sufficient cleaning efficacy provided by the cleaning fluid. For example, the vibration measurement is used to determine whether the physical interaction and/or the mechanical interaction is greater than a predetermined threshold level. If the vibration measurement does not indicate sufficient cleaning efficacy (e.g., the physical interaction and/or the mechanical interaction is less than the predetermined threshold level), additional cleaning fluid (e.g., a second cleaning fluid flow) is delivered through the lumen, in accordance with block 1152, an additional vibration measurement is obtained while the additional cleaning fluid passes through the lumen, in accordance with block 1154, and an additional determination is made regarding whether the additional vibration measurement indicates sufficient cleaning efficacy provided by the additional cleaning fluid, in accordance with block 1156. However, if the vibration measurement does indicate sufficient cleaning efficacy (e.g., the physical interaction and/or the mechanical interaction is greater than the predetermined threshold level), the lumen is flushed, such as with water, to remove any contaminant remaining in the lumen (e.g., detached by the cleaning fluid) and/or any remnants of cleaning fluid, as shown at block 1158. Flushing the lumen places the lumen in condition for any next steps in reprocessing (e.g., sterilization, drying, etc.). In this way, blocks 1152, 1154, 1156 are repeated until a sufficient cleaning efficacy is provided to the lumen, after which the lumen is flushed.

In some embodiments, the cleaning efficacy of the cleaning process is characterized by comparing a property of the vibration measurement to a threshold value. As an example, amplitudes of vibration measurements are obtained over a period of time in which the cleaning fluid is delivered through the lumen, and a peak amplitude of the vibration measurements is compared to the threshold value. The peak amplitude exceeding the threshold value can indicate sufficient cleaning efficacy of the channel cleaning process provided by the cleaning shot, whereas the peak amplitude being below the threshold value can indicate insufficient cleaning efficacy provided by the cleaning shot. As another example, quantities of vibration measurements having certain frequencies are determined. A quantity of vibration measurements having a frequency at or above a threshold frequency is determined, and such a quantity exceeding the threshold value indicates sufficient cleaning efficacy provided by the cleaning shot, whereas such a quantity being below the threshold value can indicate insufficient cleaning efficacy provided by the cleaning shot. As a further example, a measured shape of one or more vibration measurements is determined and compared to a target shape indicating sufficient cleaning efficacy. A difference between the measured shape and the target shape being below the threshold value (i.e., the measured shape and the target shape substantially match one another) can indicate sufficient cleaning efficacy provided by the cleaning shot, whereas the difference between the measured shape and the target shape being above the threshold value (i.e., the measured shape and the target shape do not substantially match one another) can indicate insufficient cleaning efficacy provided by the cleaning shot. In such an example, the target shape can be obtained during a calibration phase (e.g., in which vibration measurements of a cleaning shot providing sufficient cleaning efficacy are obtained), based on historical vibration measurements (e.g., previously obtained vibration measurements of cleaning shots providing sufficient cleaning efficacy), via a user input, using artificial intelligence, and so forth.

It should be noted that additional measurements can be used to help characterize the cleaning efficacy of the cleaning process. For example, the additional measurements can include a force imparted by the cleaning fluid against the interior surface, a speed of the cleaning fluid, a pressure in the lumen, a volume of the cleaning fluid (e.g., a clustering of cleaning shots), and/or a composition of the cleaning fluid. Indeed, certain values of the additional measurements can be more suitable for cleaning certain lumens (e.g., lumens of specific sizes), and the additional measurements can therefore be evaluated, such as compared to respective target values, to determine the suitability of the cleaning fluid for cleaning a lumen. In certain embodiments, the additional measurements can be used for changing how cleaning fluid is delivered through the lumen (e.g., to adjust the additional measurements toward the respective target values suitable for cleaning the lumen).

In embodiments in which a quantity of cleaning shots is delivered through the lumen, the vibration measurement associated with each cleaning shot is obtained and used to characterize the cleaning efficacy of each cleaning shot. Based on the characterization, a determination is made regarding a quantity of subsequent cleaning shots to be delivered through the lumen. For instance, the cleaning shots can have varying cleaning efficacies, such as a first subset of cleaning shots having sufficient cleaning efficacy and a second, remaining subset of cleaning shots having insufficient cleaning efficacy. The subsequent quantity of cleaning shots to be delivered through the lumen can be based on the second subset of cleaning shots having insufficient cleaning efficacy. As an example, the subsequent quantity of cleaning shots can match the quantity of cleaning shots in the second subset (e.g., three out of nine cleaning shots were determined to have insufficient cleaning efficacy, so three subsequent cleaning shots are subsequently delivered).

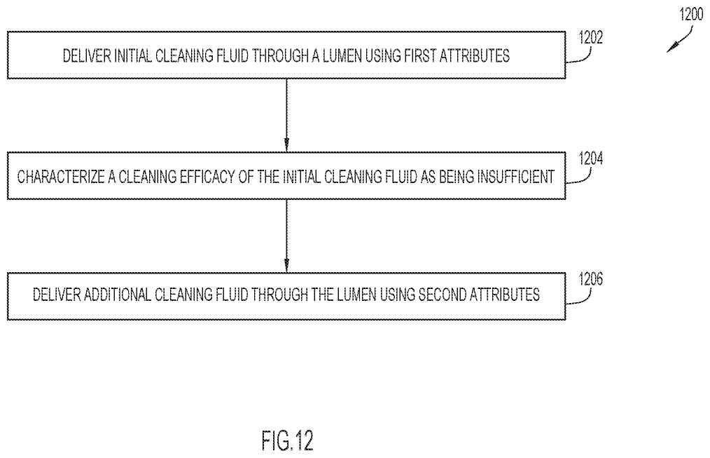

FIG. 12 is a flowchart of an embodiment of a method 1200 for cleaning a channel of a medical device by adjusting how cleaning fluid is delivered through a lumen of the channel. At block 1202, initial cleaning fluid, such as an initial cleaning shot of a fluid-powder mixture and/or an initial flow of liquid (e.g., water), is delivered through the lumen using first or initial attributes. At block 1204, the cleaning efficacy provided by the initial cleaning fluid is characterized as being insufficient. For instance, a property, such as an amplitude and/or a quantity at a certain frequency, of the vibration measurements indicative of interaction between the initial cleaning fluid and an interior surface is compared to a threshold to provide the characterization. At block 1206, in response to characterizing the cleaning efficacy provided by the initial cleaning fluid is insufficient, additional cleaning fluid, such as an additional shot of a fluid-powder mixture and/or an additional flow of liquid (e.g., water), is delivered through the lumen using second or updated attributes. In particular, the second attributes are adjusted from the first attributes. By way of example, the characterization that the cleaning efficacy provided by the initial cleaning fluid is insufficient can indicate that the first attributes used to deliver the initial cleaning fluid are not suitable for cleaning the lumen. Thus, the first attributes are adjusted to the second attributes, which can be more suitable for cleaning the lumen, and the additional cleaning fluid is produced and delivered through the lumen using the second attributes.

Each of the first attributes and second attributes can include any combination of a force imparted by cleaning fluid, a speed of cleaning fluid flow, a pressure within the lumen, a volume of cleaning fluid, and/or a composition of cleaning fluid. In certain embodiments, in response to characterizing the cleaning efficacy provided by the initial cleaning fluid as being insufficient, a value of at least one of the first attributes is compared to at least one target value to determine how the first attributes are to be adjusted. As an example, in response to determining the value is substantially different from the at least one target value, thereby indicating the initial cleaning fluid may not have been delivered as desired through the lumen, the value is adjusted toward the at least one target value to adjust the first attributes to the second attributes. For instance, in response to determining the initial cleaning fluid is delivered at a speed below the target speed, the speed is increased toward the target speed such that the additional cleaning fluid is delivered through the lumen at the increased speed. However, in additional or alternative embodiments, the first attributes are adjusted in any suitable manner, such as without comparing to any predetermined target value. Moreover, at least one attribute can be maintained between different cleaning fluids (e.g., in response to determining the attribute is at a target value). That is, one of the attributes, such as volume, of the initial cleaning fluid can match that of the additional cleaning fluid. Indeed, any of the attributes used to deliver cleaning fluid can be selectively adjusted.

In some embodiments, the cleaning efficacy of a channel cleaning process provided the additional cleaning fluid is also characterized. In response to the cleaning efficacy provided by the additional cleaning fluid being insufficient, yet another cleaning fluid can be delivered through the lumen. As an example, a subsequent cleaning fluid is delivered through the lumen using third attributes, which are adjusted from the second attributes. Thus, cleaning fluids can be repeatedly delivered through the lumen using attributes that are iteratively adjusted.

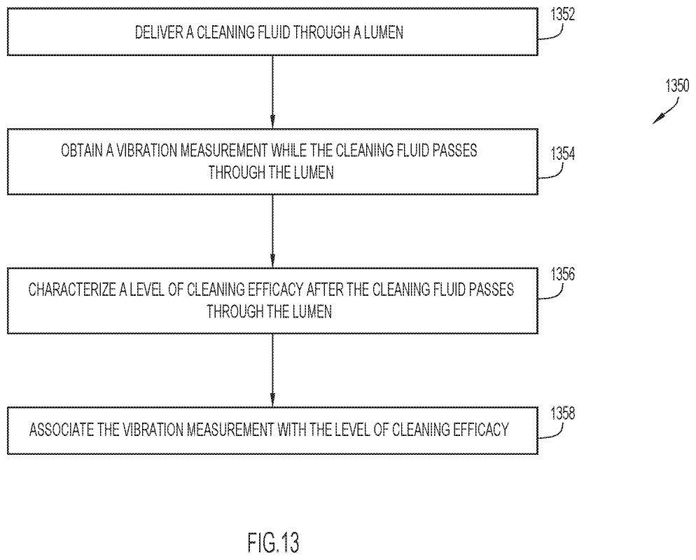

As discussed, cleaning efficacy can be characterized by comparing an obtained vibration measurement with a reference vibration measurement (e.g., by comparing a shape of the obtained vibration measurement with a shape of the reference vibration measurement). In certain embodiments, the reference vibration measurement is obtained, generated, or established by delivering cleaning fluid through a lumen. FIG. 13 is a flowchart of an embodiment of a method 1350 for obtaining such a reference vibration measurement for comparison with a subsequently obtained vibration measurement.

At block 1352, a cleaning fluid, such as a cleaning shot of a fluid-powder mixture and/or a liquid (e.g., water), is delivered through a lumen of a channel of a medical device. At block 1354, a vibration measurement is obtained while the cleaning fluid passes through the lumen. At block 1356, a level of cleaning efficacy after the cleaning fluid passes through the lumen is characterized. By way of example, an optical sensor or other observational technique is used to visually inspect the lumen to determine an amount of contaminant remaining within the lumen after the cleaning fluid has passed through the lumen. The amount of contaminant remaining within the lumen indicates the level of cleaning efficacy. In particular, an increased amount of contaminant indicates a lower level of cleaning efficacy. In certain embodiments, the amount of contaminant remaining includes a percentage of remaining contaminant after the cleaning fluid has passed through the lumen relative to a total initial amount of contaminant existing prior to the cleaning fluid passing through the lumen. At block 1358, the vibration measurement is associated with the level of cleaning efficacy.

The vibration measurement associated with the level of cleaning efficacy can then be used as reference and comparison with subsequently obtained vibration measurements, such as during performance of the method 1150. As an example, for a reference vibration measurement associated with sufficient cleaning efficacy (e.g., the determined amount of contaminant corresponding to cleaning fluid providing the reference vibration measurement is below a threshold), a subsequently obtained vibration measurement can be compared to the reference vibration measurement to determine whether the subsequently obtained vibration measurement provides sufficient cleaning efficacy. For instance, a shape, a peak amplitude, or another parameter of the subsequently obtained vibration measurement is compared to that of the reference vibration measurement associated with sufficient cleaning efficacy. A level of cleaning efficacy of the subsequently obtained vibration measurement is characterized based on the comparison (e.g., the level of cleaning efficacy is characterized as being sufficient based on the parameter of the subsequently obtained vibration measurement substantially matching that of the reference vibration measurement associated with sufficient cleaning efficacy). As another example, for a vibration measurement associated with insufficient cleaning efficacy (e.g., the determined amount of contaminant corresponding to cleaning fluid providing the reference vibration measurement is above a threshold), a subsequently obtained vibration measurement can be compared to the reference vibration measurement to determine whether the subsequently obtained vibration measurement provides insufficient cleaning efficacy (e.g., based on the parameter of the subsequently obtained vibration measurement substantially matching that of the reference vibration measurement associated with insufficient cleaning efficacy).

In certain embodiments, the method 1350 is repeatedly performed to provide multiple reference vibration measurements associated with different levels of cleaning efficacies. By way of example, cleaning fluids are delivered through the lumen using different attributes to adjust the level of cleaning efficacy provided by each cleaning fluid. In such embodiments, a subsequently obtained vibration measurement can be compared to the reference vibration measurements to characterize the particular level of cleaning efficacy of the subsequently obtained vibration measurement (e.g., based on the particular level of cleaning efficacy being associated with a reference vibration measurement that substantially matches the subsequently obtained vibration measurement). Thus, the level of cleaning efficacy provided by a cleaning fluid can be more granularly characterized.

Vibration measurements associated with a particular level of cleaning efficacy can additionally or alternatively be generated through an interpolation and/or an extrapolation technique, using artificial intelligence, etc. That is, using data that associates vibration measurements with different levels of cleaning efficacies, additional vibration measurements associated with other levels of cleaning efficacies can be determined. As such, different effective channel cleaning process signatures can be generated and used for comparison to characterize a cleaning efficacy of a channel cleaning process.

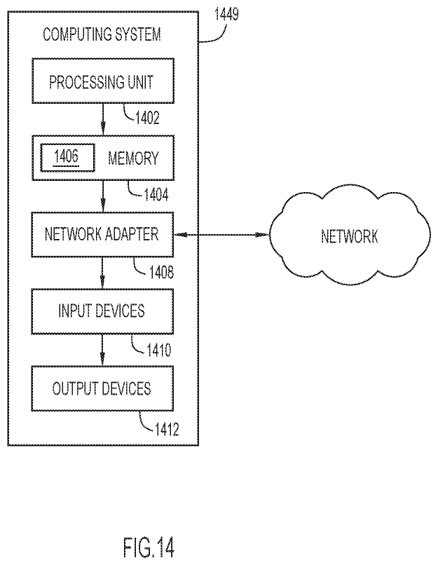

In some instances, techniques discussed herein can include, or be controlled by, a control sub-system. FIG. 14 is a block diagram illustrating an example computing device 1449 configured to operate as a control sub-system for cleaning and/or characterizing cleaning efficacy with respect to a channel of a medical device, in accordance with certain embodiments presented herein. It is to be appreciated that the arrangement for the computing device 1449 is merely illustrative, and that aspects of the techniques presented herein may be implemented at a number of different types of systems/devices. For example, the computing device 1449 can comprise a personal computer, server computer, hand-held device, tablet computer, laptop device, multiprocessor system, microprocessor-based system, programmable consumer electronic (e.g., smart phone), network PC, minicomputer, mainframe computer, tablet, remote control unit, surgical system, distributed computing environment that include any of the above systems or devices, and the like. The computing device 1449 can be a single virtual or physical device operating in a networked environment over communication links to one or more remote devices, such as an implantable medical device or implantable medical device system.

In its most basic configuration, the computing device 1449 includes at least one processing unit 1402 and memory 1404. The processing unit 1402 includes one or more hardware or software processors (e.g., Central Processing Units) that can obtain and execute instructions. The processing unit 1402 can communicate with and control the performance of other components of the computing device 1449.

The memory 1404 is one or more software or hardware-based computer-readable storage media operable to store information accessible by the processing unit 1402. The memory 1404 can store, among other things, instructions executable by the processing unit 1402 to implement applications or cause performance of operations described herein, as well as other data. The memory 1404 can be volatile memory (e.g., RAM), non-volatile memory (e.g., ROM), or combinations thereof. The memory 1404 can include transitory memory or non-transitory memory. The memory 1404 can also include one or more removable or non-removable storage devices. In examples, the memory 1404 can include RAM, ROM, EEPROM (Electronically-Erasable Programmable Read-Only Memory), flash memory, optical disc storage, magnetic storage, solid state storage, or any other memory media usable to store information for later access. In examples, the memory 1404 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 1404 can include wired media such as a wired network or direct-wired connection, and wireless media such as acoustic, RF, infrared and other wireless media, or combinations thereof. In certain embodiments, the memory 1404 comprises logic 1406 that, when executed, enables the processing unit 1402 to perform aspects of the techniques presented.

In the illustrated example, the computing device 1449 further includes a network adapter 1408 one or more input devices 1410, and one or more output devices 1412. The computing device 1449 can include other components, such as a system bus, component interfaces, a graphics system, a power source (e.g., a battery), among other components. The network adapter 1408 is a component of the computing device 1449 that provides network access (e.g., access to at least one network). The network adapter 1408 can provide wired or wireless network access and can support one or more of a variety of communication technologies and protocols, such as ETHERNET, cellular, BLUETOOTH, near-field communication, and RF (Radiofrequency), among others. The network adapter 1408 can include one or more antennas and associated components configured for wireless communication according to one or more wireless communication technologies and protocols.

The one or more input devices 1410 are devices over which the computing device 1449 receives input from a user. The one or more input devices 1410 can include physically-actuatable user-interface elements (e.g., buttons, switches, or dials), touch screens, keyboards, mice, pens, and voice input devices, among others input devices. The one or more output devices 1412 are devices by which the computing device 1449 is able to provide output to a user. The output devices 1412 can include, a display, one or more speakers, among other output devices.

As noted, merely for ease of illustration, the techniques presented herein are primarily described with reference to cleaning a specific type of medical channel, namely the channels of an endoscope. However, it will be appreciated that the disclosure is not limited to use with endoscopes or, more generally, to only use with medical devices, or with reference to fluidic composition. As such, it is to be appreciated that the techniques presented herein can be used to in association with the channels of a number of different devices/instruments used in any of a number of different applications, such as dental lines, food/drink lines, other medical channels, etc. In addition, also as noted above, aspects of the techniques presented herein can also be used with other cleaning techniques.

Certain aspects of the techniques presented herein have been described with reference to various descriptions of fluid dynamics. It is to be appreciated that these various descriptions are provided for purposes of illustration and that the innovation presented herein works regardless of the believed understanding of the fluid dynamics.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. A method, comprising:
delivering a cleaning fluid flow through a lumen of a medical device during a channel cleaning process;
obtaining a vibration measurement while the cleaning fluid flow passes through the lumen; and
characterizing a cleaning efficacy of the channel cleaning process based on the vibration measurement.

2. The method of claim 1, wherein characterizing the cleaning efficacy of the channel cleaning process comprises:
analyzing at least one of a physical or mechanical interaction between the cleaning fluid flow and the lumen.

3. The method of claim 2, wherein characterizing the cleaning efficacy of the channel cleaning process comprises:
determining whether at least one of the physical or mechanical interaction between the cleaning fluid flow and the lumen was greater than a predetermined threshold level.

4. The method of claim 3, further comprising:
delivering an additional cleaning fluid flow through the lumen in response to determining the at least one of the physical or mechanical interaction between the cleaning fluid flow and the lumen was less than the predetermined threshold level.

5. The method of claim 4, wherein the cleaning fluid flow is delivered through the lumen with one or more adjustable attributes, and wherein the method further comprises:
changing at least one of the one or more adjustable attributes to at least one updated attribute in response to determining the at least one of the physical or mechanical interaction between the cleaning fluid flow and the lumen was less than the predetermined threshold level; and
producing the additional cleaning fluid flow with the at least one updated attribute.

6. (canceled)

7. (canceled)

8. The method of claim 1, further comprising:
obtaining one or more other measurements while the cleaning fluid flow passes through the lumen; and
characterizing the cleaning efficacy of the channel cleaning process based on the vibration measurement and the one or more other measurements.

9. (canceled)

10. (canceled)

11. The method of claim 1, wherein the cleaning fluid flow comprises a fluid-powder mixture.

12. The method of claim 1, wherein characterizing the cleaning efficacy of the channel cleaning process based on the vibration measurement comprises:
comparing a peak amplitude of the vibration measurement to a threshold.

13. A system configured to perform a channel cleaning process, the system comprising:
a mechanism to deliver a cleaning fluid through at least one lumen of an apparatus;
at least one transducer operably coupled to the at least one lumen and configured to measure interaction between the cleaning fluid and the at least one lumen as the cleaning fluid passes through the at least one lumen; and
at least one processor configured to use the interaction measured by the at least one transducer to characterize a cleaning efficacy of the channel cleaning process.

14. The system of claim 13, wherein the at least one transducer is configured to capture a vibration measurement indicative of the interaction between the cleaning fluid and the at least one lumen.

15. The system of claim 13, wherein the cleaning fluid is a fluid-powder mixture.

16. The system of claim 13, wherein the cleaning fluid is water.

17. The system of claim 13, wherein the mechanism to deliver the cleaning fluid through the at least one lumen of the apparatus comprises:
a holding chamber configured to retain the cleaning fluid therein;
at least one delivery chamber fluidically connected to the at least one lumen;
at least one of a valve or pump configured to provide the cleaning fluid to the at least one delivery chamber; and
a delivery mechanism configured to apply at least one flow of fluid to the cleaning fluid in the at least one delivery chamber to propel the cleaning fluid through the at least one lumen.

18. The system of claim 13, wherein the interaction between the cleaning fluid and the at least one lumen measured by the at least one transducer comprises a plurality of vibration measurements, and the at least one processor is configured to:
determine a quantity of vibration measurements of the plurality of vibration measurements having a frequency above a threshold frequency; and
compare the quantity to a threshold quantity to characterize the cleaning efficacy of the channel cleaning process.

19. The system of claim 13, wherein the at least one transducer comprises at least one of a vibration transducer, an accelerometer, or a microphone.

20. The system of claim 13, wherein the mechanism is configured to deliver an additional cleaning fluid in response to the at least one processor characterizing the cleaning efficacy of the cleaning lumen process is insufficient.

21. (canceled)

22. (canceled)

23. The method of claim 1, further comprising:
delivering an additional cleaning fluid flow through the lumen or an additional lumen;
obtaining an additional vibration measurement while the additional cleaning fluid flow passes through the lumen or the additional lumen; and
comparing the additional vibration measurement to the vibration measurement to characterize an additional level of cleaning efficacy after the additional cleaning fluid flow passes through the lumen or the additional lumen.

24. (canceled)

25. (canceled)

26. The method of claim 1, further comprising:
apportioning an amount of cleaning fluid to provide a cleaning shot of the cleaning fluid; and
delivering the cleaning shot as the cleaning fluid flow through the lumen of the medical device.

27. The method of claim 3, comprising flushing the lumen in response to determining the at least one of the physical or mechanical interaction between the cleaning fluid flow and the lumen was greater than the predetermined threshold level.

28. The method of claim 1, wherein the vibration measurement comprises a vibration generated as a result of an interaction between the cleaning fluid flow and a wall of the lumen during movement of the cleaning fluid flow through the lumen.
